(19) United States
(12) Patent Application Publication
(10) Pub. No.: US 2012/0027732 A1
(43) Pub. Date: Feb. 2, 2012

(54) THERMOREVERSIBLE COLLAGEN

(76) Inventor: Sherry L. Voytik-Harbin, Zionsville, IN (US)

(21) Appl. No.: 13/192,276

(22) Filed: Jul. 27, 2011

Related U.S. Application Data
(60) Provisional application No. 61/368,162, filed on Jul. 27, 2010.

(51) Int. Cl. A61K 38/39; A61P 17/02; A61K 35/12; A61P 17/00; C07K 14/78; A61K 35/00

(52) U.S. Cl. 424/93.7; 530/356; 424/93.1; 514/17.2

(57) ABSTRACT

This invention relates to collagen formulations. More particularly, this invention relates to thermoreversible collagen formulations and methods of their use and preparation.

This application claims the benefit of and priority to U.S. Provisional Patent Application Ser. No. 61/368,162, filed Jul. 27, 2010, the disclosure of which is incorporated herein by reference.

### TECHNICAL FIELD

This invention relates to the field of collagen formulations. More particularly, this invention relates to thermoreversible collagen formulations and methods of their use.

### BACKGROUND AND SUMMARY OF THE INVENTION

Collagen is the most abundant protein in the body, presenting many biological signals and maintaining the mechanical integrity of many different tissues. Collagen has the ability to self-associate in vitro, forming gels that can act as a three-dimensional substrate, and provide mechanical and biological signals for cell growth. Traditionally, collagens are used as a matrix for continuous culture of cells within a three-dimensional format. However, the removal of cells requires enzyme-mediated (e.g., collagenase) degradation of the matrix. The requirement to use enzymes to harvest cells from collagen may be time consuming and may be undesirable in some in vivo applications.

The present invention provides thermoreversible collagen formulations that reversibly transition between solution and matrix phases in response to temperature modulation between 4° C. and 37° C. The thermoreversible collagen formulations can be substantially free of enzymes and can facilitate the rapid and complete dissolution of the collagen matrix. A natural collagen polymer matrix is ideally suited as a three-dimensional cell culture substrate and cryopreservative medium for cells since it provides functional cell-adhesion motifs in a physiologically-relevant fibril context. At 4° C., for example, the thermoreversible collagen is in a solution phase. A subsequent increase in temperature to 37° C., for example, induces polymerization of the collagen matrix comprising insoluble collagen fibrils and the matrix may further comprise an interstitial fluid phase. Following the increase in temperature, if the temperature is decreased, the collagen matrix can then deploymerize. Subsequent cycles of polymerization and deplolymerization can occur. The thermoreversible collagen of the present invention is biocompatible and can be used for the propagation of cells.

The following embodiments of the invention are contemplated but are non-limiting:

1. A composition comprising an engineered collagen matrix wherein the matrix comprises reduced collagen and wherein the composition further comprises a population of cells.

2. The composition of clause 1 wherein the reduced collagen consists essentially of reduced collagen oligome

3. The composition of clause 1 wherein the reduced collagen consists essentially of reduced collagen monomers.

4. The composition of any one of clauses 1 to 3 wherein the cells are selected from the group consisting of hematopoietic stem cells, endothelial progenitor cells, mesenchymal stem cells, dermal fibroblasts, keratinocytes, chondrocytes, epithelial cells, osteoblasts, fibroblasts, smooth muscle cells, cardiac muscle cells, hepatocytes, skin cells, lung cells, cells of the ovary, and cells of the colon.

5. The composition of clause 4 wherein the cells are endothelial progenitor cells.

6. The composition of clause 4 wherein the cells are hematopoietic stem cells.

7. The composition of any one of clauses 1 to 2 or 4 to 6 wherein the collagen comprises oligomer 260 collagen.

8. The composition of any one of clauses 1 to 7 wherein the collagen is derived from porcine skin.

9. The composition of any one of clauses 1 to 8 wherein the collagen is thermoreversible collagen.

10. The composition of any one of clauses 1 to 9 wherein the reduced collagen comprises reduced reactive aldehydes.

11. The composition of any one of clauses 1 to 10 for use as a drug delivery device.

12. The composition of any one of clauses 1 to 10 for use as a cell delivery device.

13. The composition of any one of clauses 1 to 10 for use as a thermoreversible matrix that can be injected into a patient for wound healing.

14. The composition of any one of clauses 1 to 10 for use as a matrix for the in vitro expansion and isolation of the cells.

15. The composition of any one of clauses 1 to 14 wherein the cells are stem cells or progenitor cells.

16. The composition of any one of clauses 1 to 10 for use as a graft construct for implantation into a patient.

17. A method for expanding and isolating a population of cells for implantation into a patient, the method comprising the steps of
seeding a polymerized, engineered collagen matrix with a population of cells wherein the matrix comprises collagen consisting essentially of reduced collagen;
expanding the population of cells;
depolymerizing the matrix;
separating the cells from the depolymerized collagen; and

implanting the cells into the patient.

18. The method of clause 17 wherein the cells are selected from the group consisting of hematopoietic stem cells, endothelial progenitor cells, mesenchymal stem cells, dermal fibroblasts, keratinocytes, chondrocytes, epithelial cells, osteoblasts, fibroblasts, smooth muscle cells, cardiac muscle cells, hepatocytes, skin cells, lung cells, cells of the ovary, and cells of the colon.

19. The method of clause 17 wherein the reduced collagen consists essentially of reduced collagen oligomers.

20. The method of clause 17 wherein the reduced collagen consists essentially of reduced collagen monomers.

21. The method of clause 18 wherein the cells are endothelial progenitor cells.

22. The method of clause 18 wherein the cells are hematopoietic stem cells.

23. The method of clause 17 wherein the collagen comprises oligomer 260 collagen.

24. The method of any one of clauses 17 to 23 wherein the collagen is derived from porcine skin.

25. The method of any one of clauses 17 to 24 wherein the collagen is thermoreversible collagen.

26. The method of any one of clauses 17 to 25 wherein the collagen matrix is polymerized by heating to 37° C.

27. The method of any one of clauses 17 to 26 wherein the collagen matrix is depolymerized by cooling to 4° C.

28. The method of any one of clauses 17 to 26 wherein the collagen matrix is depolymerized by enzymatic dissolution.

29. The method of clause 28 wherein the enzyme is collagenase.

30. The method of any one of clauses 17 to 29 wherein the cells are separated from the collagen by centrifugation.

31. The method of any one of clauses 17 to 30 wherein the collagen is reduced using sodium borohydride.

32. The method of any one of clauses 17 to 31 wherein the cells are separated from the collagen by centrifugation in a cell harvest buffer.

33. The method of clause 32 wherein the cell harvest buffer comprises a sugar and a calcium chelator.

34. The method of clause 33 wherein the sugar is glucose.

35. The method of clause 33 or 34 wherein the calcium chelator is EDTA.

36. The method of any one of clauses 17 to 35 wherein the collagen is reduced in a buffer at a neutral pH.

37. The method of any one of clauses 17 to 19 or 21 to 36 wherein the collagen comprises oligomer 260 collagen.

38. A composition comprising an engineered collagen matrix wherein the matrix comprises collagen consisting essentially of atelopeptide collagen and the composition further comprises a population of cells.

39. The composition of clause 38 wherein the cells are selected from the group consisting of hematopoietic stem cells, endothelial progenitor cells, mesenchymal stem cells, dermal fibroblasts, keratinocytes, chondrocytes, epithelial cells, osteoblasts, fibroblasts, smooth muscle cells, cardiac muscle cells, hepatocytes, skin cells, lung cells, cells of the ovary, and cells of the colon.

40. The composition of clause 39 wherein the cells are endothelial progenitor cells.

41. The composition of clause 39 wherein the cells are hematopoietic stem cells.

42. The composition of any one of clauses 38 to 41 wherein the collagen is derived from porcine skin.

43. The composition of any one of clauses 38 to 42 wherein the collagen is thermoreversible collagen.

44. The composition of any one of clauses 38 to 43 for use as a drug delivery device.

45. The composition of any one of clauses 38 to 43 for use as a cell delivery device.

46. The composition of any one of clauses 38 to 43 for use as a thermoreversible matrix that can be injected into a patient for wound healing.

47. The composition of any one of clauses 38 to 43 for use as a matrix for the in vitro expansion and isolation of the cells.

48. The composition of any one of clauses 38 to 43 for use as a graft construct for implantation into a patient.

49. The composition of any one of clauses 38 to 48 further comprising reduced collagen oligomers.

50. The composition of any one of clauses 38 to 48 further comprising reduced collagen monomers.

51. The composition of any one of clauses 38 to 48 further comprising oligomer 260 collagen.

52. A composition comprising an engineered collagen matrix wherein the matrix comprises sterilized thermoreversible collagen.

53. The composition of clause 52 further comprising cells.

54. The composition of clause 52 wherein the thermoreversible collagen consists essentially of reduced collagen monomers.

55. The composition of clause 52 wherein the thermoreversible collagen comprises reduced collagen oligomers.

56. The composition of clause 55 wherein the percentage of collagen oligomers based on total isolated collagen in dry weight/ml used to make the matrix is about 10 percent or greater.

57. The composition of clause 53 wherein the cells are hematopoietic stem cells.

58. The composition of clause 52 wherein the composition is in a sterile package.

59. The composition of clause 58 wherein the composition is a medical graft.

60. The composition of clause 55 wherein the composition is in a sterile package and wherein the composition is a medical graft.

61. A method for isolating cells for implantation into a patient, the method comprising the steps of,
polymerizing thermoreversible collagen to form an engineered collagen matrix;
contacting the thermoreversible collagen with the cells before or after polymerizing the thermoreversible collagen to form the matrix;
proliferating the cells or maintaining the viability of the cells;
depolymerizing the matrix comprising thermoreversible collagen;
separating the cells from the depolymerized thermoreversible collagen; and
implanting or injecting the cells into the patient.

62. The method of clause 61 wherein the thermoreversible collagen consists essentially of reduced collagen monomers.

63. The method of clause 61 wherein the thermoreversible collagen comprises reduced collagen oligomers.

64. The method of clause 63 wherein the percentage of collagen oligomers based on total isolated collagen in dry weight/ml used to make the matrix is about 10 percent or greater.

65. The method of clause 61 wherein the cells are hematopoietic stem cells.

66. The method of clause 61 wherein the cells are used as an injectable or implantable composition for wound healing, a bone marrow transplant, or for cosmetic surgery.

67. The method of clause 61 wherein the polymerization occurs in response to heating to a temperature that causes a collagen solution to matrix transition and wherein the depolymerization occurs in response to cooling to a temperature that causes a collagen matrix to solution transition.

68. A method for treating a patient with diseased or damaged tissues, the method comprising the step of implanting or injecting thermoreversible collagen into the patient.

69. The method of clause 68 wherein the thermoreversible collagen consists essentially of reduced collagen monomers.

70. The method of clause 68 wherein the thermoreversible collagen comprises reduced collagen oligomers.

71. The method of clause 68 wherein the thermoreversible collagen is in the form of an injectable or an implantable medical graft for wound healing or for cosmetic surgery.

### BRIEF DESCRIPTION OF THE DRAWINGS

FIG. 1 shows a schematic of example collagen building blocks which comprise oligomer (A), monomer (B), reduced-oligomer (C), reduced-monomer (D), and pepsin-treated atelo-peptide (E) collagen formulations.

FIG. 2 shows an SDS-PAGE gel (4%) of collagen formulations after selective elimination of intermolecular cross-links. An oligomer 260 band is shown in the oligomer preparation.

FIG. 3 shows various collagen formulations that differ in the type and content of intermolecular cross-links produce collagen matrices with different viscoelastic properties.

FIG. 4 shows the time-dependent changes in shear storage modulus (G′) for oligomer (upper line) and reduced-oligomer (lower line) collagens in response to temperature modulation between 37° C. and 4° C.

FIG. 5 shows the time-dependent changes in shear storage modulus (G′) for monomer (upper line) and reduced-monomer (lower line) collagens in response to temperature modulation between 37° C. and 4° C.

FIG. 6 shows the time-dependent changes in shear storage modulus (G′) for atelo-collagen in response to temperature modulation between 37° C. and 4° C.

FIG. 7 shows the time-dependent changes in absorbance at 405 nm (A405) for oligomer (upper line) and reduced-oligomer (lower line) collagens in response to temperature modulation between 37° C. and 4° C.

FIG. 8 shows the time-dependent changes absorbance at 405 nm (A405) for monomer (upper line) and reduced-monomer (lower line) collagens in response to temperature modulation between 37° C. and 4° C.

FIG. 9 shows the time-dependent changes absorbance at 405 nm (A405) for atelo-collagen in response to temperature modulation between 37° C. and 4° C.

FIG. 10 shows a comparison of LSK (hematopoietic stem cells) proliferation within various collagen culture parameters (oligomer preparations with various storage moduli) in the presence or absence of osteoblasts.

FIG. 11 shows a comparison of LSK viability within various collagen culture parameters (oligomer preparations with various storage moduli) in the presence or absence of osteoblasts.

FIG. 12 shows a comparison of the percentage of LSK Lin− Sca1+ cells within various collagen culture parameters (oligomer preparations with various storage moduli) in the presence or absence of osteoblasts.

FIG. 13 shows confocal reflection images showing single slice (1 um) views of collagen-fibril microstructure of oligomer and reduced-oligomer matrices.

FIG. 14 shows a thermoreversible type I collagen formulation (upper panel) compared to unmodified collagen formulation (lower panel).

FIG. 15 shows Matrix physical properties (e.g., G′) can be varied by modulating relevant polymerization parameters (e.g., collagen volume fraction).

FIG. 16 shows the application of thermoreversible collagen for continuous 3D culture and/or cryopreservation of cells.

FIG. 17 shows the viability and recovery efficiency of VM-2, NHDF, and EPC-Artery cultured within 3D thermoreversible collagen matrix (G′=150 Pa) (upper panels); and the light micrographs of 3D cell cultures after 48 hours (bottom panels).

### DETAILED DESCRIPTION OF THE ILLUSTRATIVE EMBODIMENTS

As used herein “engineered collagen matrix” means a matrix that is polymerized in vitro under predetermined conditions selected from the group consisting of, but not limited to, pH, phosphate concentration, temperature, buffer composition, ionic strength, and composition and concentration of the collagen. An “engineered collagen matrix” can be made from purified collagen or partially purified extracellular matrix components.

As used herein “partially purified extracellular matrix components” are extracellular matrix components that are solubilized from intact extracellular matrix material wherein the collagen in the “partially purified extracellular matrix components” is not substantially free from impurities.

As used herein “purified collagen” is collagen that is substantially free of impurities (e.g., collagen that is 95% to 99.9% pure).

As used herein “engineered purified collagen matrix” means a purified collagen-based matrix that is polymerized in vitro under predetermined conditions selected from the group consisting of, but not limited to, pH, phosphate concentration, temperature, buffer composition, ionic strength, and composition and concentration of the collagen. An “engineered purified collagen matrix” is made from purified collagen.

As used herein “engineering a matrix” means polymerizing an “engineered collagen matrix” or an “engineered purified collagen matrix” in vitro.

As used herein “thermoreversible collagen” means collagen that can reversibly transition between solution and matrix phases in response to temperature modulation between 4° C. and 37° C. or temperature modulation between any other temperatures that cause reversible matrix to solution transitions.

As used herein “reduced collagen” means collagen that is reduced in vitro to eliminate or substantially reduce reactive aldehydes. For example, collagen may be reduced in vitro by treatment of collagen with a reducing agent (e.g., sodium borohydride).

As used herein “atelopeptide collagen” means collagen that is treated in vitro with pepsin or another suitable protease or agent to eliminate or substantially reduce telopeptide regions which contain intermolecular cross-linking sites.

As used herein “oligomer 260 collagen” is a collagen preparation made (e.g., from porcine skin), by procedures resulting in isolation of oligomers, where the collagen preparation has a prominent band at molecular weight 260, where the band is not prominent or is lacking in corresponding monomer preparations. The presence of the band can be determined by SDS polyacrylamide gel electrophoresis.

As used herein “hematopoietic stem cells” means hematopoietic stem cells and associated progenitor cells. Hematopoietic stem cells can be identified and/or isolated based on specific cell markers (e.g., the Lineage−, Sca1+, and c-Kit+ hematopoietic stem cell markers) or specific functions characteristic of hematopoietic stem cells and known to those skilled in the art.

In one embodiment, a composition comprising an engineered collagen matrix is provided where the matrix comprises reduced collagen and where the composition further comprises a population of cells. In another embodiment, a method for expanding and isolating a population of cells for implantation into a patient is provided. The method comprises the steps of seeding a polymerized, engineered collagen matrix with a population of cells wherein the matrix comprises collagen consisting essentially of reduced collagen, expanding the population of cells, depolymerizing the matrix, separating the cells from the depolymerized collagen, and implanting the cells into the patient.

In these method and composition embodiments, the collagen can be derived from porcine skin, and the collagen can comprise reduced collagen. In various illustrative embodiments, the composition can be for use as a drug delivery device (e.g., a device where a drug is reversibly attached to collagen and is released over time due to collagen degradation in vivo), for use as a cell delivery device, for use as a thermoreversible matrix that can be injected into a patient for wound healing or for other therapeutic or cosmetic applications such as plastic surgery, for use as a matrix for the in vitro expansion and isolation (e.g., separation of the cells from the collagen) of the cells, for use in cryopreservation (e.g., to prevent ice crystal formation upon freezing of cells at low temperature), for use in generating tissue constructs for implantation into a patient after separation of the tissue constructs from the collagen (e.g., vessels using, for example, endothelial progenitor cells grown on the matrices), or for use as a graft construct for implantation into a patient.

In these method and composition embodiments, the reduced collagen can comprise or consist essentially of reduced collagen oligomers, can comprise or consist essentially of reduced collagen monomers, or can comprise oligomer 260 collagen. The cells can be selected from the group consisting of hematopoietic stem cells, endothelial progenitor cells, mesenchymal stem cells, dermal fibroblasts, keratinocytes, chondrocytes, epithelial cells, osteoblasts, fibroblasts, smooth muscle cells, cardiac muscle cells, hepatocytes, skin cells, lung cells, cells of the ovary, and cells of the colon. In these embodiments, the collagen can be thermoreversible.

In the method embodiment, the collagen can be polymerized by heating to 37° C., the collagen matrix can be depolymerized by cooling to 4° C. or by heating or cooling to any other temperature that causes a reversible transition between solution (e.g., depolymerized collagen) and matrix (e.g., polymerized collagen) phases, the collagen matrix can be depolymerized by enzymatic dissolution, the enzyme for dissolution can be collagenase, and the cells can be separated from the collagen, for example, by centrifugation. In the method embodiment, the collagen can be reduced using sodium borohydride, the cells can be separated from the collagen by centrifugation in a cell harvest buffer, the cell harvest buffer can comprise a sugar and a calcium chelator, the sugar can be glucose, the calcium chelator can be EDTA, and the collagen can be reduced in a buffer at a neutral pH.

In another illustrative embodiment, a composition is provided comprising an engineered collagen matrix wherein the matrix comprises collagen comprising or consisting essentially of atelopeptide collagen and the composition further comprises a population of cells. In this composition embodiment, the collagen can be derived from porcine skin. The composition can be for use as a drug delivery device, for use as a cell delivery device, for use as a thermoreversible matrix that can be injected into a patient for wound healing or for other therapeutic or cosmetic applications such as plastic surgery, for use as a matrix for the in vitro expansion and isolation of the cells, for use in cryopreservation, or for use as a graft construct for implantation into a patient. In this composition embodiment, the cells can be selected from the group consisting of hematopoietic stem cells, endothelial progenitor cells, mesenchymal stem cells, dermal fibroblasts, keratinocytes, chondrocytes, epithelial cells, osteoblasts, fibroblasts, smooth muscle cells, cardiac muscle cells, hepatocytes, skin cells, lung cells, cells of the ovary, and cells of the colon. In these embodiments, the collagen can be thermoreversible.

In another embodiment, a composition is provided comprising an engineered collagen matrix wherein the matrix comprises sterilized thermoreversible collagen. In various embodiments, the composition can further comprise cells, can include thermoreversible collagen consisting essentially of reduced collagen monomers, or can include thermoreversible collagen comprising reduced collagen oligomers. In the embodiment where the matrix comprises reduced collagen oligomers, the percentage of collagen oligomers based on total isolated collagen in dry weight/ml used to make the matrix can be about 10 percent or greater, 15 percent or greater, 12 percent or greater, or 8 percent or greater, for example. The cells can be of any type described herein including hematopoietic stem cells. The composition can be in a sterile package and can be a medical graft.

In yet another embodiment, a method for isolating cells for implantation into a patient is provided. The method comprises the steps of polymerizing thermoreversible collagen to form an engineered collagen matrix, contacting the thermoreversible collagen with the cells before or after polymerizing the thermoreversible collagen to form the matrix, proliferating the cells or maintaining the viability of the cells, depolymerizing the matrix comprising thermoreversible collagen, separating the cells from the depolymerized thermoreversible collagen, and implanting the cells into the patient. In various embodiments, the thermoreversible collagen can consist essentially of reduced collagen monomers, or can comprise reduced collagen oligomers. In the embodiment where the matrix comprises reduced collagen oligomers, the percentage of collagen oligomers based on total isolated collagen in dry weight/ml used to make the matrix can be about 10 percent or greater, 15 percent or greater, 12 percent or greater, or 8 percent or greater, for example. The cells can be of any type described herein including hematopoietic stem cells, or any cells suitable for implantation into a patient. The cells can be used for injection or implantation, for example, for wound healing or for cosmetic surgery applications. The polymerization can occur in response to heating to a temperature that causes a collagen solution to matrix transition, and the depolymerization can occur in response to cooling to a temperature that causes a collagen matrix to solution transition.

In another illustrative embodiment, a method for treating a patient with diseased or damaged tissues is provided. The method comprises the step of implanting or injecting thermoreversible collagen into the patient. In various embodiments, the thermoreversible collagen can consist essentially of reduced collagen monomers, or can comprise reduced collagen oligomers. In the embodiment where the matrix comprises reduced collagen oligomers, the percentage of collagen oligomers based on total isolated collagen in dry weight/ml used to make the matrix can be about 10 percent or greater, 15 percent or greater, 12 percent or greater, or 8 percent or greater, for example. Cells can be implanted with the thermoreversible collagen and can be of any type described herein including hematopoietic stem cells, or any cells suitable for implantation into a patient. The cells can be used for injection or implantation, for example, for wound healing or for cosmetic surgery applications.

In another embodiment, a method for preparing a graft construct is provided. The method comprises the steps of polymerizing thermoreversible collagen to form an engineered collagen matrix, and implanting the matrix into the patient. The method can further comprise the steps of contacting the thermoreversible collagen with cells before or after polymerizing the thermoreversible collagen to form the matrix, and proliferating the cells or maintaining the viability of the cells. In various embodiments, the thermoreversible collagen can consist essentially of reduced collagen monomers, or can comprise reduced collagen oligomers. In the embodiment where the matrix comprises reduced collagen oligomers, the percentage of collagen oligomers based on total isolated collagen in dry weight/ml used to make the matrix can be about 10 percent or greater, 15 percent or greater, 12 percent or greater, or 8 percent or greater, for example. The cells can be of any type described herein including hematopoietic stem cells, or any cells suitable for implantation into a patient. The matrix or the cells can be used for injection or implantation, for example, for wound healing, for bone marrow transplants, or for cosmetic surgery applications.

All of the embodiments described below apply to any embodiment described in the preceding nine paragraphs, or to any embodiment (i.e., clauses or other embodiments) of the invention described in the Summary section of this application. In any of these embodiments, the thermoreversible collagen can be used to culture and expand a population of cells on the matrix in vitro, depolymerize the matrix to separate the matrix from the cells, and to then implant the cells into a patient in need of treatment with the cells.

In any embodiment described herein, the engineered collagen matrix can be prepared by utilizing acid-solubilized collagen and defined or predetermined polymerization conditions that are controlled to yield three-dimensional collagen matrices with a range of controlled assembly kinetics (e.g., polymerization half-time), molecular compositions, and fibril microstructure-mechanical properties, for example, as described in U.S. patent application Ser. Nos. 11/435,635 (published Nov. 22, 2007, as Publication No. 2007-0269476 A1) and 11/903,326 (published Oct. 30, 2008, as Publication No. 2008-0268052), each incorporated herein by reference.

In one aspect, purified collagen or partially purified extracellular matrix components can be used and can be obtained from a number of sources, including for example, porcine skin, to construct the engineered collagen matrices described herein. Suitable tissues useful as a collagen-containing source material for isolating collagen or extracellular matrix components to make the engineered collagen matrices described herein are submucosa tissues or any other extracellular matrix-containing tissues of a warm-blooded vertebrate. Suitable methods of preparing submucosa tissues are described in U.S. Pat. Nos. 4,902,508; 5,281,422; and 5,275,826, each incorporated herein by reference. Extracellular matrix material-containing tissues other than submucosa tissue may be used to obtain collagen in accordance with the methods and compositions described herein. Sources or methods of preparing other extracellular matrix material-derived tissues for use in obtaining purified collagen or partially purified extracellular matrix components are known to those skilled in the art. For example, see U.S. Pat. Nos. 5,163,955 (pericardial tissue); 5,554,389 (urinary bladder submucosa tissue); 6,099,567 (stomach submucosa tissue); 6,576,265 (extracellular matrix tissues generally); 6,793,939 (liver basement membrane tissues); and U.S. patent application publication no. US-2005-0019419-A1 (liver basement membrane tissues); and international publication no. WO 2001/45765 (extracellular matrix tissues generally), each incorporated herein by reference. In various other embodiments, the collagen-containing source material can be selected from the group consisting of placental tissue, ovarian tissue, uterine tissue, animal tail tissue, and skin tissue. Any suitable extracellular matrix-containing tissue can be used as a collagen-containing source material to isolate purified collagen or partially purified extracellular matrix components.

An illustrative preparation method for preparing submucosa tissues as a source of purified collagen or partially purified extracellular matrix components is described in U.S. Pat. No. 4,902,508, the disclosure of which is incorporated herein by reference. In one embodiment, a segment of vertebrate intestine, for example, preferably harvested from porcine, ovine or bovine species, but not excluding other species, is subjected to abrasion using a longitudinal wiping motion to remove cells or cell-removal is accomplished by hypotonic or hypertonic lysis. In one embodiment, the submucosa tissue is rinsed under hypotonic conditions, such as with water or with saline under hypotonic conditions and is optionally sterilized. In another illustrative embodiment, such compositions can be prepared by mechanically removing the luminal portion of the tunica mucosa and the external muscle layers and/or lysing resident cells with hypotonic or hypertonic washes, such as with water or saline. In these embodiments, the submucosa tissue can be stored in a hydrated or dehydrated state prior to isolation of the purified collagen or partially purified extracellular matrix components. In various aspects, the submucosa tissue can comprise any delamination embodiment, including the tunica submucosa delaminated from both the tunica muscularis and at least the luminal portion of the tunica mucosa of a warm-blooded vertebrate.

In the various embodiments described herein, the purified collagen can also comprise exogenously added glycoproteins, proteoglycans, glycosaminoglycans (e.g., chondroitins and heparins), hyaluronic acid, etc. In another embodiment, the partially purified extracellular matrix components can comprise glycoproteins, proteoglycans, glycosaminoglycans (e.g., chondroitins and heparins), hyaluronic acid, etc. extracted from the insoluble fraction with the collagen.

In various illustrative embodiments, the purified collagen or the partially purified extracellular matrix components or the engineered collagen matrices formed from these components can be disinfected and/or sterilized prior to seeding the matrices with the cells, using conventional sterilization techniques including propylene oxide or ethylene oxide treatment, gas plasma sterilization, gamma radiation, electron beam, and/or peracetic acid sterilization. Sterilization techniques which do not adversely affect the structure and biotropic properties of the collagen can be used. Illustrative sterilization techniques are exposing the purified collagen or the partially purified extracellular matrix components or the engineered collagen matrices to peracetic acid, 1-4 Mrads gamma irradiation (or 1-2.5 Mrads of gamma irradiation), ethylene oxide treatment, or gas plasma sterilization. In one embodiment, the collagen-containing source material, the purified collagen, the partially purified extracellular matrix components, or the engineered collagen matrices can be subjected to one or more sterilization processes. In an illustrative embodiment, peracetic acid can be used for sterilization.

Typically, prior to extraction, the collagen-containing source material is comminuted by tearing, cutting, grinding, or shearing the collagen-containing source material. In one illustrative embodiment, the collagen-containing source material can be comminuted by shearing in a high-speed blender, or by grinding the collagen-containing source material in a frozen state (e.g., at a temperature of −20° C., −40° C., −60° C., or −80° C. or below prior to or during the comminuting step) and then lyophilizing the material to produce a powder having particles ranging in size from about 0.1 mm² to about 1.0 mm². In one illustrative embodiment, the collagen-containing source material is comminuted by freezing and pulverizing under liquid nitrogen in an industrial blender. In this embodiment, the collagen-containing source material can be frozen in liquid nitrogen prior to, during, or prior to and during the comminuting step.

In any of the illustrative embodiments described herein, after comminuting the collagen-containing source material, the material can be mixed (e.g., by blending or stirring) with an extraction solution to extract and remove soluble proteins. Illustrative extraction solutions include sodium acetate (e.g., 0.5 M and 1.0 M). Other methods for extracting soluble proteins are known to those skilled in the art and are described in detail in U.S. Pat. No. 6,375,989, incorporated herein by reference. Illustrative extraction excipients include, for example, chaotropic agents such as urea, guanidine, sodium chloride or other neutral salt solutions, magnesium chloride, and non-ionic or ionic surfactants.

In any illustrative aspect described herein, after the initial extraction, the soluble fraction can be separated from the insoluble fraction to obtain the insoluble fraction. For example, the insoluble fraction can be separated from the soluble fraction by centrifugation (e.g., 2000 rpm at 4° C. for 1 hour). In alternative embodiments, other separation techniques known to those skilled in the art, such as filtration, can be used. In one embodiment, the initial extraction step can be repeated one or more times, discarding the soluble fractions. In another embodiment, after completing the extractions, one or more steps can be performed of washing the insoluble fraction with water, followed by centrifugation, and discarding the supernatant.

In any of the embodiments described herein, the insoluble fraction can then be extracted (e.g., with 0.075 M sodium citrate) to obtain the purified collagen or the partially purified extracellular matrix components. In illustrative aspects the extraction step can be repeated multiple times retaining the soluble fractions. In one embodiment, the accumulated soluble fractions can be combined and can be clarified to form the soluble fraction, for example by centrifugation (e.g., 2000 rpm at 4° C. for 1 hour).

In any embodiment described herein, the soluble fraction can be fractionated to isolate the purified collagen, or the partially purified extracellular matrix components. In one illustrative aspect, the soluble fraction can be fractionated by dialysis. Suitable molecular weight cut-offs for the dialysis tubing or membrane are from about 3,500 to about 12,000 or about 3,500 to about 5,000 or about 12,000 to about 14,000. In various illustrative embodiments, the fractionation, for example by dialysis, can be performed at about 2° C. to about 37° C. for about 1 hour to about 96 hours. In one embodiment, the soluble fraction is dialyzed against a buffered solution (e.g., 0.02 M sodium phosphate dibasic). However, the fractionation can be performed at any temperature, for any length of time, and against any suitable buffered solution. In one embodiment, the precipitated collagen-containing material is then collected by centrifugation (e.g., 2000 rpm at 4° C. for 1 hour). In another embodiment, one or more steps can be performed of washing the collagen-containing material with water, followed by centrifugation, and discarding the supernatant.

In any of the embodiments described herein, the collagen-containing material can then be resuspended in an aqueous solution wherein the aqueous solution is acidic. For example, the aqueous acidic solution can be an acetic acid solution, but any other acids including hydrochloric acid, formic acid, lactic acid, citric acid, sulfuric acid, ethanoic acid, carbonic acid, nitric acid, or phosphoric acid can be used. For example, acids, at concentrations of from about 0.001 N to about 0.1 N, from about 0.005 N to about 0.1 N, from about 0.01 N to about 0.1 N, from about 0.05 N to about 0.1 N, from about 0.001 N to about 0.05 N, from about 0.001 N to about 0.01 N, or from about 0.01 N to about 0.05 N can be used to resuspend the collagen-containing material. In another embodiment the collagen-containing material can be resuspended in water.

The term “lyophilized” means that water is removed, completely or partially, from the composition, typically by freeze-drying under a vacuum. In one illustrative aspect, the isolated resuspended collagen-containing material can be lyophilized after it is resuspended for storage. In another illustrative embodiment, a matrix can be formed and the engineered collagen matrix itself can be lyophilized for storage. In one illustrative lyophilization embodiment, the resuspended collagen-containing material is first frozen, and then placed under a vacuum. In another lyophilization embodiment, the resuspended collagen-containing material can be freeze-dried under a vacuum. In another lyophilization embodiment, the collagen-containing material can be lyophilized before resuspension. Any method of lyophilization known to the skilled artisan can be used.

In any of the embodiments described herein, the acids described above can be used as adjuvants for storage after lyophilization in any combination. The acids that can be used as adjuvants for storage include hydrochloric acid, acetic acid, formic acid, lactic acid, citric acid, sulfuric acid, ethanoic acid, carbonic acid, nitric acid, or phosphoric acid, and these acids can be used at any of the above-described concentrations. In one illustrative embodiment, the lyophilizate can be stored (e.g., lyophilized in and stored in) an acid, such as acetic acid, at a concentration of from about 0.001 N to about 0.5 N or from about 0.01 N to about 0.5 N. In another embodiment, the lyophilizate can be stored in water with a pH of about 6 or below. In another embodiment, the lyophilized product can be stored dry. In other illustrative embodiments, lyoprotectants, cryoprotectants, lyophilization accelerators, or crystallizing excipients (e.g., ethanol, isopropanol, mannitol, trehalose, maltose, sucrose, tert-butanol, and tween 20), or combinations thereof, and the like can be present during lyophilization.

In any of the illustrative embodiments described herein, the collagen-containing material can be directly sterilized after resuspension, for example, with peracetic acid or with peracetic acid and ethanol (e.g., by the addition of 0.18% peracetic acid and 4.8% ethanol to the resuspended collagen-containing material before lyophilization). In another embodiment, sterilization can be carried out during the fractionation step. For example, the collagen-containing material can be dialyzed against chloroform, peracetic acid, or a solution of peracetic acid and ethanol (e.g., 0.18% peracetic acid and 4.8% ethanol) to disinfect or sterilize the material. The chloroform, peracetic acid, or peracetic acid/ethanol can be removed prior to lyophilization, for example by dialysis against an acid, such as 0.01 N acetic acid. In an alternative embodiment, the lyophilized composition can be sterilized directly after rehydration, for example, by the addition of 0.18% peracetic acid and 4.8% ethanol. In this embodiment, the sterilizing agent can be removed prior to polymerization of the collagen to form fibrils.

In any embodiment described herein, the collagen-containing material can be dialyzed against 0.01 N acetic acid, for example, prior to lyophilization to remove the sterilization solution and so that the collagen is in a 0.01 N acetic acid solution. In another embodiment, the collagen-containing material can be dialyzed against hydrochloric acid, for example, prior to lyophilization and can be lyophilized in hydrochloric acid and redissolved in hydrochloric acid, acetic acid, or water.

For use in producing the engineered collagen matrix that can be used for the maintenance, proliferation (i.e., expansion), differentiation, or culture of cells or their progeny, the redissolved lyophilizate can be subjected to varying conditions (e.g., pH, phosphate concentration, temperature, buffer composition, ionic strength, and composition and concentration of the purified collagen (dry weight/ml) or partially purified extracellular matrix components (dry weight/ml)) that result in polymerization to form an engineered collagen matrix with specific characteristics.

In any of the illustrative embodiments described herein, as discussed above, the polymerization reaction for the engineered collagen matrices can be conducted in a buffered solution using any biologically compatible buffer system known to those skilled in the art. For example, the buffer may be selected from the group consisting of phosphate buffer saline (PBS), Tris (hydroxymethyl)aminomethane Hydrochloride (Tris-HCl), 3-(N-Morpholino) Propanesulfonic Acid (MOPS), piperazine-n,n′-bis(2-ethanesulfonic acid) (PIPES), [n-(2-Acetamido)]-2-Aminoethanesulfonic Acid (ACES), N-[2-hydroxyethyl]piperazine-N′-[2-ethanesulfonic acid] (HEPES) and 1,3-bis[tris (Hydroxymethyl)methylamino]propane (Bis Tris Propane). In one embodiment the buffer is PBS, Tris, or MOPS and in one embodiment the buffer system is PBS, and more particularly 10×PBS. In accordance with one embodiment, the 10×PBS buffer at pH 7.4 comprises the following ingredients:

1.37 M NaCl

0.027 M KCl

0.081 M Na₂HPO₄

0.015 M KH₂PO₄

5 mM MgCl₂

55.5 mM glucose

All of the conditions that can be varied to polymerize and engineer the matrices described herein (e.g., pH, phosphate concentration, temperature, buffer composition, ionic strength, and composition and concentration of the collagen-containing material (dry weight/ml)) are described in U.S. application Ser. No. 11/903,326 (published Oct. 30, 2008, as Publication No. 2008-0268052), incorporated herein by reference.

The collagen can be polymerized either in the presence of cells or cells can be added to an already polymerized collagen matrix. Thermoreversible collagen can be polymerized or depolymerized in the presence of cells. After depolymerization, the collagen and cells can be separated easily (e.g., by centrifugation) to separate the cells from the collagen without the need for treatment of the collagen with an enzyme. The polymerization results in transition to the matrix phase. The depolymerization results in transition to the solution phase.

The purified collagen and the partially purified extracellular matrix components are derived from a collagen-containing source material and, in some embodiments, may contain glycoproteins, such as laminin and fibronectin, proteoglycans, such as serglycin, versican, decorin, and perlecan, and glycosaminoglycans. In one embodiment, the collagen in the collagen-containing source material can be purified or partially purified to isolate the collagen using protocols known to the skilled artisan. In these embodiments, the purified collagen can be about 95%, about 96%, about 97%, about 98%, or about 99% pure, for example. In other embodiments, the purified collagen can be from about 95% to about 99.9% pure, from about 96% to about 99.9% pure, or from about 97% to about 99.9% pure. In yet another illustrative embodiment, the phrase “purified collagen” means the isolation of collagen in a form that is substantially free from impurities (e.g., typically the total amount of other components present in the composition represents less than 5%, or more typically less than 0.1%, of total dry weight). In an alternate embodiment, purified collagen can be purchased from sources such as Sigma Chemical Co. (St. Louis, Mo.), Advanced BioMatrix, Inc. (San Diego, Calif.), or Nutacon (Leimuiden, Netherlands).

As discussed, the engineered collagen matrices as herein described may be made under controlled conditions to obtain particular mechanical properties. For example, the engineered collagen matrices may have desired collagen fibril density, pore size (fibril-fibril branching), elastic modulus, tensile strain, tensile stress, linear modulus, compressive modulus, loss modulus, fibril area fraction, fibril volume fraction, collagen concentration, cell seeding density, shear storage modulus (G′ or elastic (solid-like) behavior), and phase angle delta (δ or the measure of the fluid (viscous)- to solid (elastic)-like behavior; δ equals 0° for Hookean solid and 90° for Newtonian fluid).

As used herein, a “modulus” can be an elastic or linear modulus (defined by the slope of the linear region of the stress-strain curve obtained using conventional mechanical testing protocols; i.e., stiffness), a compressive modulus, a loss modulus, or a shear storage modulus (e.g., a storage modulus). These terms are well-known to those skilled in the art.

As used herein, a “fibril volume fraction” (i.e., fibril density) is defined as the percent area of the total area occupied by fibrils in three dimensions.

As used herein, tensile or compressive stress “σ” is the force carried per unit of area and is expressed by the equation:

$$σ = \frac{P}{A} = \frac{P}{ab}$$

where:
   s=stress
   P=force
   A=cross-sectional area
   a width
   h=height
      The force (P) produces stresses normal (i.e., perpendicular) to the cross section of the part (e.g., if the stress tends to lengthen the part, it is called tensile stress, and if the stress tends to shorten the part, it is called compressive stress).

As used herein, “tensile strain” is the strain caused by bending and/or stretching a material.

In any embodiment described herein, the fibril volume fraction of the matrix can be about 1% to about 60%. In various embodiments, the engineered collagen matrix can contain fibrils with specific characteristics, for example, a fibril volume fraction of about 2% to about 60%, about 2% to about 40%, about 5% to about 60%, about 15% to about 60%, about 2% to about 30%, about 5% to about 30%, about 15% to about 30%, or about 20% to about 30%.

In any of the illustrative embodiments described herein, the engineered collagen matrix can contain fibrils with specific characteristics, including, but not limited to, a modulus (e.g., a compressive modulus, loss modulus, or a storage modulus) of about 10 Pa to about 50000 Pa, about 10 Pa to about 10000 Pa, about 10 Pa to about 5000 Pa, about 10 Pa to about 3000 Pa, about 10 Pa to about 2000 Pa, about 10 Pa to about 1000 Pa, about 10 Pa to about 700 Pa, about 10 Pa to about 300 Pa, about 10 Pa to about 200 Pa, about 10 Pa to about 100 Pa, about 500 Pa to about 2000 Pa, about 700 Pa to about 1500 Pa, about 700 Pa to about 900 Pa, or about 800 Pa. In any of the embodiments described herein, the matrices made with oligomeric collagen can have enhanced stiffness compared to matrices made with monomeric collagen.

In any of the embodiments described herein, the engineered collagen matrix can contain fibrils with specific characteristics, including, but not limited to, a phase angle delta (δ) of about 0° to about 12°, about 0° to about 5°, about 1° to about 5°, about 4° to about 12°, about 5° to about 7°, about 8° to about 10°, and about 5° to about 10°.

In any of the illustrative embodiments described herein, qualitative and quantitative microstructural characteristics of the engineered collagen matrices can be determined by environmental or cryostage scanning electron microscopy, transmission electron microscopy, confocal microscopy, second harmonic generation multi-photon microscopy. In another embodiment, tensile, compressive and viscoelastic properties can be determined by rheometry or tensile testing. All of these methods are known in the art or are further described in U.S. patent application Ser. No. 11/435,635 (published Nov. 22, 2007, as Publication No. 2007-0269476 A1), or are described in Roeder et al., J. Biomech. Eng., vol. 124, pp. 214-222 (2002), in Pizzo et al., J. Appl. Physiol., vol. 98, pp. 1-13 (2004), Fulzele et al., Eur. J. Pharm. Sci., vol. 20, pp. 53-61 (2003), Griffey et al., J. Biomed. Mater. Res., vol. 58, pp. 10-15 (2001), Hunt et al., Am. J. Surg., vol. 114, pp. 302-307 (1967), and Schilling et al., Surgery, vol. 46, pp. 702-710 (1959), incorporated herein by reference.

In another embodiment, a method for preparing the compositions described herein comprising an engineered collagen matrix and cells is provided. In this embodiment, the method comprises the steps of engineering the matrix comprising collagen fibrils, and contacting the matrix with cells. In this embodiment, the matrix can be prepared from reduced collagen or atelopeptide collagen (e.g., reduced collagen oligomers, reduced collagen monomers, atelopeptide collagen, or reduced or non-reduced oligomer 260 collagen).

Typically, the engineered collagen matrices are prepared from isolated collagen at collagen concentrations ranging from about 0.05 mg/ml to about 5.0 mg/ml, about 1.0 mg/ml to about 3.0 mg/ml, about 0.1 mg/ml to about 4.0 mg/ml, about 0.5 mg/ml to about 3.5 mg/ml, about 0.5 mg/ml to about 5.0 mg/ml, about 0.05 mg/ml to about 10 mg/ml, or about 0.05 to about 20 mg/ml, for example. In various illustrative embodiments, the collagen concentration is about 0.3 mg/ml, about 0.5 mg/ml, about 0.75 mg/ml, about 1.0 mg/ml, about 1.5 mg/ml, about 2.0 mg/ml, about 2.5 mg/ml, about 3.0 mg/ml, about 3.5 mg/ml, or about 5.0 mg/ml.

In any of these embodiments the engineered collagen matrix is seeded with the cells. In various embodiments, the engineered collagen matrix can be seeded with one or more cell types in combination. In one illustrative embodiment, osteoblasts and hematopoietic stem cells can be added and the osteoblasts can enhance proliferation, maintenance, or function of the hematopoietic stem cells. The engineered collagen matrix can be seeded with autogenous cells isolated from the patient to be treated. In an alternative embodiment the cells may be xenogeneic or allogeneic in nature.

In any of the embodiments described herein, the cells can be seeded on the engineered collagen matrix at a cell density of about 1×10⁶ to about 1×10⁸ cells/ml, or at a density of about 1×10³ to about 2×10⁶ cells/ml. In one embodiment, cells are seeded at a density of less than 5×10⁴ cells/ml. In another embodiment cells are seeded at a density of less than 1×10⁴ cells/ml. In another embodiment, cells are seeded at a density selected from a range of about 1×10² to about 5×10⁶, about 0.3×10⁴ to about 60×10⁴ cells/ml, and about 0.5×10⁴ to about 50×10⁴ cells/ml. Any suitable cell density can be used. The cells are maintained, proliferated, differentiated, and/or cultured according to methods described herein or to methods well-known to the skilled artisan for cell culture.

In any of the various embodiments described herein, the engineered collagen matrices of the present invention can be combined, prior to, during, or after polymerization, with nutrients, including minerals, amino acids, sugars, peptides, proteins, vitamins (such as ascorbic acid), or glycoproteins that facilitate cell culture, proliferation, differentiation, and/or maintenance, such as laminin and fibronectin, hyaluronic acid, or growth factors such as platelet-derived growth factor, or transforming growth factor beta, and glucocorticoids such as dexamethasone. In other illustrative embodiments, fibrillogenesis inhibitors, such as glycerol, glucose, or polyhydroxylated compounds can be added prior to or during polymerization of the matrix. In accordance with one embodiment, cells can be added to the purified collagen or the partially purified extracellular matrix components as the last step prior to the polymerization or after polymerization of the engineered collagen matrix. In other illustrative embodiments, cross-linking agents, such as carbodiimides, aldehydes, lysl-oxidase, N-hydroxysuccinimide esters, imidoesters, hydrazides, and maleimides, and the like can be added before, during, or after polymerization.

In any of the embodiments described herein, the cells may be isolated from the matrix, using an enzyme, for subsequent injection or implantation into a patient. For example, cells can be isolated from the matrix using collagenase or a solution thereof. Additional enzymes useful for isolation of cells from the matrix include, for example, proteases such as serine proteases, thiol proteases, and metalloproteinases, including the matrix metalloproteinases such as the collagenases, gelatinases, stromelysins, and membrane type metalloproteinase, or combinations thereof.

In any of the embodiments described herein, the collagen used herein may be any type of collagen, including collagen types Ito XXVIII, alone or in any combination. In one embodiment, a mixture of type I and type III collagen is used. In one illustrative embodiment, the type III collagen can enhance differentiation and proliferation of the cells seeded on the engineered collagen matrices.

In any of the embodiments described herein, the cells can be suspended in a liquid-phase, collagen formulation designed to polymerize in situ to form a three-dimensional matrix. The formulation can comprise soluble collagen, for example, soluble type I collagen, and defined polymerization reaction conditions to yield engineered collagen matrices with controlled molecular composition, fibril microstructure, and mechanical properties (e.g., stiffness), for example. Matrix stiffness and fibril density can predictably modulate cell behavior.

Applicants have developed type I collagen formulations derived from various collagen sources, e.g., pig skin. These formulations comprise both type I collagen monomers (single triple helical molecules) and oligomers (at least two monomers covalently crosslinked together). The presence of oligomers enhances the self-assembly potential by increasing the assembly rate and by yielding three-dimensional matrices with distinct fibril microstructures and increased mechanical integrity (e.g., stiffness).

In any of the embodiments described herein, the engineered collagen matrix can have a predetermined percentage of collagen monomers or oligomers or oligomer 260 collagen based on total isolated collagen (dry weight/ml) added to make the engineered matrix. In various embodiments, the predetermined percentage of collagen monomers or oligomers or oligomer 260 collagen can be about 10% or more, about 15% or more, about 12% or more, about 0.5% to about 100%, about 30% to about 100%, about 40% to about 100%, about 50% to about 100%, about 60% to about 100%, about 70% to about 100%, about 80% to about 100%, about 90% to about 100%, about 95% to about 100%, or about 100%. In yet another embodiment, the collagen oligomers are obtained from a collagen-containing source material enriched with collagen oligomers (e.g., pig skin).

In any of the embodiments described herein, the engineered collagen matrices can have an oligomer content quantified by average polymer molecular weight (AMW). As described herein, modulation of AMW can affect polymerization kinetics, fibril microstructure, molecular properties, and fibril architecture of the matrices, for example, interfibril branching, pore size, and mechanical integrity (e.g., matrix stiffness). In another embodiment, the oligomer content of the purified collagen, as quantified by average polymer molecular weight, positively correlates with matrix stiffness.

In any of the embodiments described herein, monomer-rich collagen matrices can have an AMW of about 100 to about 280 kDa, about 250 to about 280 kDa, or about 250 to about 300 kDa, e.g., about 282 kDa. In another illustrative embodiment, oligomer-rich collagen matrices have an AMW of greater than about 300 kDa, for example, the AMW of an oligomer-rich collagen matrix can be about 300 kDa to about 2.8 MDa, about 400 kDa to about 2.8 MDa, about 400 kDa to about 750 kDa, about 400 kDa to about 850 kDa, about 350 kDa to about 1.5 MDa, or about 350 kDa to about 2.0 MDa. In one embodiment, the oligomer-rich collagen matrices have an AMW of greater than about 2.8 MDa.

In one embodiment, the thermoreversible collagen comprising reduced collagen monomers, reduced collagen oligomers, or atelopeptide collagen, or a combination, can be in a frozen solution, for example, a packaged frozen solution. The solution can be sterilized and the package can be a sterile package, such as a sterile vial.

The following examples illustrate specific embodiments in further detail. These examples are provided for illustrative purposes only and should not be construed as limiting the invention or the inventive concept in any way.

### Example 1

### Collagen Isolation

All type I collagen formulations were prepared from the dermis of market weight pigs. Type I collagen, comprising oligomers and monomers, was acid solubilized and purified from porcine skin according to a modified protocol from (Gallop, P. M. and S. Seifter, Preparation and properties of soluble collagens, Methods in Enzymology, 1963, p. 635-641, incorporated herein by reference). All type I collagen formulations were prepared from the dermis of market weight pigs. To prepare collagen, skin was harvested from pig immediately following euthanasia and was washed thoroughly with cold water. The skin was stretched out and pinned to a board and stored at 4° C. The hair was removed with clippers. The dermal layer of the tissue was isolated by separating and removing the upper epidermal layer and the lower loose fatty connective layers. This removal was readily achieved by scraping the tissue with a knife or straight razor. The tissue was maintained at 4° C.

The resulting dermal layer tissue was washed in water and then cut into small pieces (approximately 1 cm²) and was frozen and stored at 80° C. The frozen skin pieces were pulverized under liquid nitrogen using an industrial blender or cryogenic grinder. Oligomer collagen was prepared as described previously (Kreger et al., Biopolymers, vol. 93, pp. 690-707, 2010, incorporated herein by reference).

Soluble proteins were removed by extracting the pig skin powder (0.125 g/ml) with 0.5M sodium acetate overnight at 4° C. The resulting mixture was then centrifuged at 2000 rpm (700×g) at 4° C. for 1 hour. The supernatant was discarded and the extraction procedure repeated three additional times. The resulting pellet was then suspended (0.25 g/ml) in cold MilliQ water and then centrifuged at 2000 rpm (700×g) at 4° C. for 1 hour. The pellet was then washed with water two additional times. Collagen extraction was then performed by suspending the pellet (0.125 g/ml) in 0.075 M sodium citrate. The extraction was allowed to proceed for 15-18 hours at 4° C. The resulting mixture was centrifuged at 2000 rpm (700×g) at 4° C. for 1 hour. The supernatant was retained and stored at 4° C. The pellet was re-extracted with 0.075 M sodium citrate. The extraction process was repeated such that the tissue was extracted a total of three times. The resulting supernatants were then combined and centrifuged at 9750 rpm (17,000×g) at 4° C. for 1 hour to clarify the solution. The supernatant was retained and the pellet discarded.

Collagen was then precipitated from the supernatant by dialyzing (MWCO 12-14,000) extensively against 0.02 M disodium hydrogen phosphate at 4° C. The resulting suspension was then centrifuged at 2000 rpm at 4° C. for 1 hour and the pellet retained. The pellet was then resuspended and rinsed in cold MilliQ water. The suspension was centrifuged at 2000 rpm at 4° C. for 1 hour. The water rinse procedure was repeated two additional times. The resulting collagen pellet was dissolved in 0.1 M acetic acid and then lyophilized. The lyophilized material was stored within a dessicator at 4° C. for use in engineering collagen matrices.

In one embodiment, selective polymerization in the presence of glycerol was used to further fractionate the pig skin collagen into oligomer-rich formulations as described previously (Na G. C., Biochemistry, 1989; 28(18):7161-7, incorporated herein by reference). A single source was obtained by performing the glycerol separation on isolated collagen obtained from a single pig hide. A pooled source was obtained from two collagen isolation batches from each of three separate pigs. Viscoelastic properties of polymerized matrices were measured in both oscillatory shear and unconfined compression on a stress-controlled AR2000 rheometer (TA Instruments, New Castle, Del.) using a stainless steel 40 mm diameter parallel plate geometry as described previously (Kreger S. T. et al., Matrix Biol, 2009; 28(6):336-46, incorporated herein by reference). Compared to the monomer-rich fraction, the oligomer-rich fraction showed a sharp increase in G′ and Ec as a function of concentration while maintaining a consistently low 8. An increase in G′, decrease in δ, and surprising increase in Ec was observed. These results indicate that changes in the fibril microstructure-mechanical properties observed with increased average molecular weight (AMW) are primarily due to increased interfibril cross-linking.

Collagen monomer was prepared by washing the tissue in 4.5 M NaCl, 50 mM Tris, pH 7.5 followed by extraction in 0.5 M acetic acid. Salt precipitation (Brennan and Davison, 1980) then was used to selectively eliminate or minimize oligomers from the monomer formulation. The resulting solution was dialyzed exhaustively against 0.1M acetic acid and lyophilized.

Reduced collagens were processed to eliminate reactive aldehydes generated from acid-labile cross-links. Here, neutral-buffered solutions of collagen oligomer and monomer solutions (1 mg/ml) were chemically reduced by stirring with sodium borohydride (1 mg/10 mg collagen). Fresh sodium borohydride was added at 30 minute intervals for a total reduction time of 90 minutes (Gelman, Williams, and Piez J. Biol. Chem. 1979). Reduced collagen solutions were then dialyzed extensively against 0.1M acetic acid and then lyophilized. To eliminate telopeptide regions which contain intermolecular cross-linking sites, collagens (2 mg/ml) were enzymatically digested in 0.5 M acetic acid containing 0.1 mg/ml pepsin at 4° C. After 24 hours, fresh pepsin was added (0.1 mg/ml) and the solution incubated at 4° C. for an additional 24 hours. All collagens were dialyzed extensively against 0.1 M acetic acid and then lyophilized. Prior to use, lyophilized collagens were dissolved in 0.01 N HCl. For cell studies, collagens were rendered aseptic by exposure to chloroform overnight at 4° C. Collagen concentration was determined using a Sirius Red (Direct Red 80) assay as previously described.

Sodium dodecyl sulfate polyacrylamide gel electrophoresis (SDS-PAGE) was used to assess the purity and molecular composition of each collagen source. 12% Novex Tris-Glycine gels (Invitrogen, Carlsbad, Calif.) were used for identification of non-collagenous proteins and small molecular weight contaminants. SDS-PAGE in interrupted and uninterrupted formats and western blot analysis using mouse monoclonal antibodies specific for type I (AB6308, Abcam, Cambridge, Mass.) and type III (MAB 1343, Chemicon, Temecula) collagen were used for analysis of collagen type content (e.g. types I, III, and V). Gels were stained with Coomassie Blue (Sigma-Aldrich) or silver nitrate and imaged using a digital camera and light box. An alcian blue assay was used as previously described to assess sulfated glycosaminoglycan (GAG) content. Heparin derived from porcine intestinal mucosa (Sigma-Aldrich) was used to prepare a standard curve (1-20 heparin units/ml).

### Example 2

### Preparation of Three-Dimensional Collagen Matrices

All collagen preparations were polymerized under identical reaction conditions to produce three-dimensional matrices as described previously (Kreger et al., 2010). Collagen solutions were diluted with 0.01 N HCl and neutralized with 10× phosphate buffered saline (PBS, 1×PBS had 0.17 M total ionic strength and pH 7.4) and 0.1 N sodium hydroxide to achieve neutral pH (7.4). Neutralized collagen solutions were kept on ice prior to the induction of polymerization by warming to 37° C. Due to the increased viscosity of collagen solutions, positive displacement pipettes (Microman, Gilson, Inc., Middleton, Wis.) were used to accurately pipette all collagen solutions.

### Example 3

### Analysis of Matrix to Solution Properties

Polymerization and depolymerization of neutralized collagen samples were monitored based upon changes in turbidity or viscoelastic properties as measured in oscillatory shear. Turbidity measurements were conducted using a Lambda 3B UV-VIS spectrophotometer (Perkin-Elmer) equipped with a temperature-controlled, 6-position holder. Absorbance measurements at 405 nm were recorded at 10 second intervals as each sample was subjected to the following warming-cooling cycle: 37° C. for 15 minutes, 4° C. for 30 minutes, and 37° C. for 15 minutes. Viscoelastic properties were measured by loading samples in oscillatory shear using an AR2000 rheometer (TA Instruments, New Castle, Del.) adapted with a stainless steel 40 mm diameter parallel plate geometry. Neutralized collagen solutions (1 ml) were pipetted onto the testing plate which was equilibrated to 4° C. Viscoelastic properties were measured at 1% strain and 1 Hz (chosen from predetermined linear viscoelastic response range). Measurements were obtained at 10-second intervals as the samples were subjected to the following warming-cooling cycle: 37° C. for 15 minutes, 4° C. for 30 minutes, and 37° C. for 30 minutes. The controlling software calculated shear storage (G′, elastic/solid component representing stored, recoverable energy) and loss (G″, viscous/fluid component representing energy permanently lost during deformation) moduli, which are related by phase shift (δ) as tan(G″/G′).

### Example 4

### Analysis of Collagen Polymerization Kinetics

A turbidimetric assay was used to analyze the polymerization (fibrillogenesis) kinetics of each collagen source as described previously (Brightman et al., 1999). Kinetic parameters calculated from the sigmoidal-shaped turbidity curves included lag time (x-intercept of line tangent to the inflection point of the sigmoidal turbidity curve), polymerization rate during growth phase (slope averaged around inflection point), maximum absorbance value, and polymerization half-time (time at which absorbance equals half the maximum absorbance value).

### Example 5

### Analysis of Collagen Fibril Microstructure

Collagen matrices were polymerized (2 h in 37° C. humidified incubator) in Lab-Tek IV chambered coverglass slides (Nunc, Thermo Fisher Scientific, Rochester, N.Y.) and overlaid with PBS. Confocal reflection microscopy (CRM) was used to collect high resolution 3D images of the matrices in their native, hydrated state. Confocal imaging was performed on an Olympus Fluoview FV1000 confocal system adapted to an Olympus IX81 inverted microscope with a 60× UPlanSApo water immersion objective (Olympus, Tokyo, Japan).

### Example 6

### Analysis of Matrix Mechanical Properties

Viscoelastic properties of polymerized collagen matrices were measured in both oscillatory shear and compression on a stress-controlled AR2000 rheometer (TA Instruments, New Castle, Del.) using a stainless steel 40 mm diameter parallel plate geometry as described previously (Kreger et al., 2010). Following polymerization of samples, a shear strain sweep from 0.01 to 5% strain at 1 Hz (chosen from predetermined linear viscoelastic response regions) was used to measure the shear modulus (reported values are at 1% strain). The controlling software calculated shear storage (G′, elastic/solid component representing stored, recoverable energy) and loss (G″, viscous/fluid component representing energy permanently lost during deformation) moduli, which are related by phase shift (3) as tan (G″/G′). Following the strain sweep, compressive behavior of each sample was evaluated in an unconfined format. Normal force was measured in response to compressive strain generated by depressing the geometry at a rate of 20 μm/s (strain rate 2.76%/s). Stress-strain plots were generated for each sample, with compressive strain calculated as 1−L/L₀ (Cauchy or engineering strain, L=height and L₀=initial height) and stress calculated as normal force divided by plate area. The compressive modulus (E_{c}) was calculated using linear regression of the slope of the stress-strain curve from approximately 15 to 60% strain. Shear and compression tests were performed on 3 independent matrices per matrix formulation (n=3).

### Example 7

### Cell Harvest from Tissue Constructs

Cells were isolated from 3D tissue construct using enzymatic or non-enzymatic dissolution of the matrix. Enzymatic digestion involved incubation of tissue constructs in complete medium containing 500 U/ml collagenase (Worthington, Type IV) and 2.4 U/ml dispase for 20 minutes at 37° C. Following digestion, an equal volume of complete medium was added and the cell suspension centrifuged at 1000 rpm for 5 minutes. The pellet was washed in complete medium and then treated with 100 ul TrypLE (Gibco) for 15 minutes at 37° C. The cell suspension was diluted in complete medium, centrifuged to concentrate, and resuspended in complete medium.

For tissue constructs exhibiting temperature-dependent matrix to solution properties, cells were isolated in ice-cold cell harvest buffer containing 1 mM EDTA, 10% w/v glucose in phosphate buffered saline, pH 7.4. Constructs in cell harvest buffer were maintained at 4° C. for 10 minutes with periodic agitation and then centrifuged at 1000 rpm for 5 minutes. The cell pellet was redissolved in complete medium.

### Example 8

### Animal Examples

Adult B6.5JL-PtγcqPep3b/BoyJ (BoyJ) mice (6-8 weeks old), C57BL/6 mice (2 day pups and 6-8 weeks old), C57BL/6×BoyJ F1 mice (6-8 weeks old) were used. Mice were bred and housed in the animal facility at Indiana University.

### Example 9

### Preparation of Osteoblasts (OB)

Calvarial OB were prepared following a modification of published methods. Calvariae from C57BL/6 mice less than 48 hours old were dissected, pretreated with EDTA in PBS for 30 minutes then subjected to sequential collagenase digestions (200 U/mL). Fractions 3-5 (collected between 45-60 minutes, 60-75 minutes, and 75-90 minutes through the digestion) were collected and used as OB. These cells are >95% OB or OB precursors as previously demonstrated.

### Example 10

### Cell Staining and Flow Cytometry

Cells were washed once with stain wash (PBS, 1% BCS, and 1% Penicillin-streptomycin) followed by antibody staining for 15 minutes on ice. Cells were washed with cold stain wash after each step.

For LSK cell sorting and phenotyping, Lin− Sca1+cKit+ (LSK) cells were sorted on BD FACS Aria. Cells harvested from co-cultures were stained with the above Ab combinations along with pacific blue (PB)-conjugated CD45.1 and PE-Cy7-conjugated CD45.2. CD45.1+ cells were gated and analyzed for the presence of Lin− Sca1+ cells on a BD LSRII. Since cultured cells quickly loose the expression of c-Kit, they were not analyzed for CD117.

### Example 11

### OB-LSK Co-Culture Set-Up

LSK (625 cells) from BoyJ mice (CD45.1) were seeded alone or in the presence of freshly isolated calvarial OB (25,000 cells) from C57B1/6 mice (CD45.2) within oligomer and reduced-oligomer collagen matrices prepared with G′ values of 150 Pa and 800 Pa (0.5 ml/well of 24-well plate). Parallel experiments were set in 2D on tissue culture plastic and involved seeding densities of 500 LSK/well and 20,000 OB/well within a 24-well plate. Cultures were maintained for one week in medium consisting of 1:1 mix of IMDM and αMEM supplemented with 10% FBS, 1% Pen/Strep, and 1% L-Glutamine. All cultures were supplemented with a cocktail of cytokines containing recombinant murine SCF & IL3 (10 ng/mL), IGF1 & TPO (20 ng/mL), IL6 & Flt3 (25 ng/mL) and OPN (50 ng/mL) on day 0 and every 2 days thereafter. Cells were harvested on day 7 using either enzymatic or non-enzymatic methods and counted. Fold increase in the number of cells derived from LSK cells was calculated relative to day 0 count.

### Example 12

### Progenitor Cell Assay

Cells were plated in duplicate in 3 cm Petri dishes containing 1 ml methyl-cellulose with cytokines (MethoCult GF M3434, Stem Cell Technologies, Vancouver, BC). Cultures were maintained at 37° C. in humidified incubator at 5% CO₂ and colonies were counted on an inverted microscope after 7 days.

### Example 13

### Examples of Collagen Building Blocks

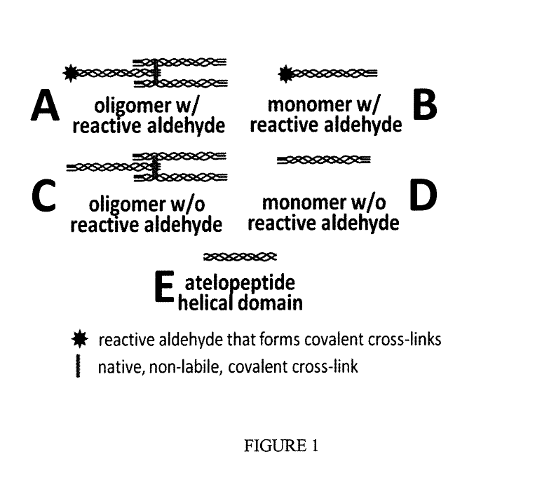

As shown in FIG. 1, collagen building blocks comprise oligomer (A), monomer (B), reduced-oligomer (C), reduced-monomer (D), and pepsin-treated atelo-(E) collagen formulations. Acid-solubilization of pig skin collagen yields a mixture of oligomers (two or more collagen molecules joined by a stable covalent intermolecular cross-links) and monomers (individual collagen molecules). The type of oligomer may vary depending upon the tissue source. Upon acid solubilization, a subset of cross-links that are acid-labile are converted to reactive aldehydes. These aldehydes react spontaneously with available amine groups to reform covalent cross-links upon polymerization. Chemical reduction of oligomer and monomer collagens with sodium borohydride eliminates reactive aldehydes. Pepsin digestion of acid-solubilized collagens selectively cleaves telopeptide regions from the amino- and carboxy-terminus of the collagen molecule thereby leaving only the triple helical domain and eliminating stable intermolecular cross-links and reactive aldehydes.

### Example 14

### SDS-PAGE Analysis of Collagen Formulations

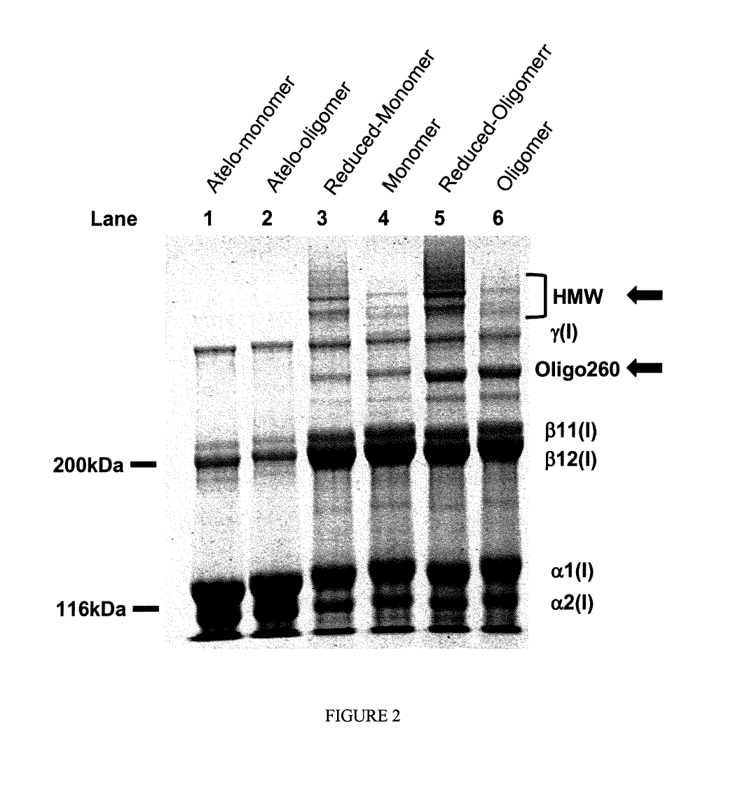

As shown in FIG. 2, a SDS-PAGE gel (4%) of collagen formulations was prepared after selective elimination of intermolecular cross-links. Oligomer (Lane 6) contains prominent protein bands corresponding to molecular weights of 260 KDa (Oligo 260) and greater than 300 KDa (HMW) in addition to the expected α1(I), α2(I), β11(I), β12(I) and γ(I) bands routinely observed in denatured collagen preparations. Oligo 260 and HMW components were found at substantially reduced levels in the monomer (Lane 4). Sodium borohydride reduction of monomer (Lane 3; reduced-monomer) and oligomer (Lane 5; reduced-oligomer) results in little to no effect on the protein banding pattern. Pepsin removal of telopeptide regions from monomer (Lane 1; atelo-monomer) and oligomer (Lane 2; atelo-oligomer) show an expected increase in mobility of α1 and α2 chains, reduction of) β11 and β12 levels, and elimination of Oligo260 and HMW components. Molecular weight markers are indicated in the left margin of FIG. 2.

### Example 15

### Intermolecular Cross-Links of Collagen Affect Viscoelastic Properties

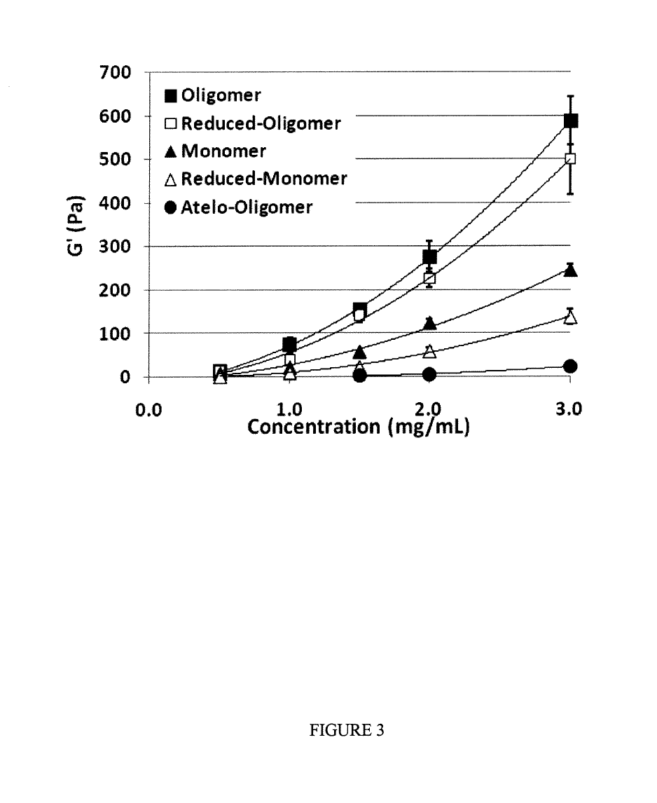

Collagen formulations that differ in the type and content of intermolecular cross-links produce collagen matrices with different viscoelastic properties are shown in FIG. 3. Collagen formulations were polymerized under identical reaction conditions at collagen concentrations of 0.5-3 mg/ml. Following polymerization of samples, a shear strain sweep from 0.01 to 5% strain at 1 Hz (chosen from predetermined linear viscoelastic response regions) was used to measure the shear storage modulus (G′; values reported at 1% strain, 1 Hz frequency (n=3)). G′ increased with increasing collagen concentration, but showed distinct relationships for each source. Collectively, these results suggest that reactive aldehydes and stable intermolecular cross-links contribute uniquely to the viscoelastic properties of polymerized collagen matrices.

### Example 16

### Oligomer and Reduced-Oligomer Collagen Changes in Shear Storage Modulus

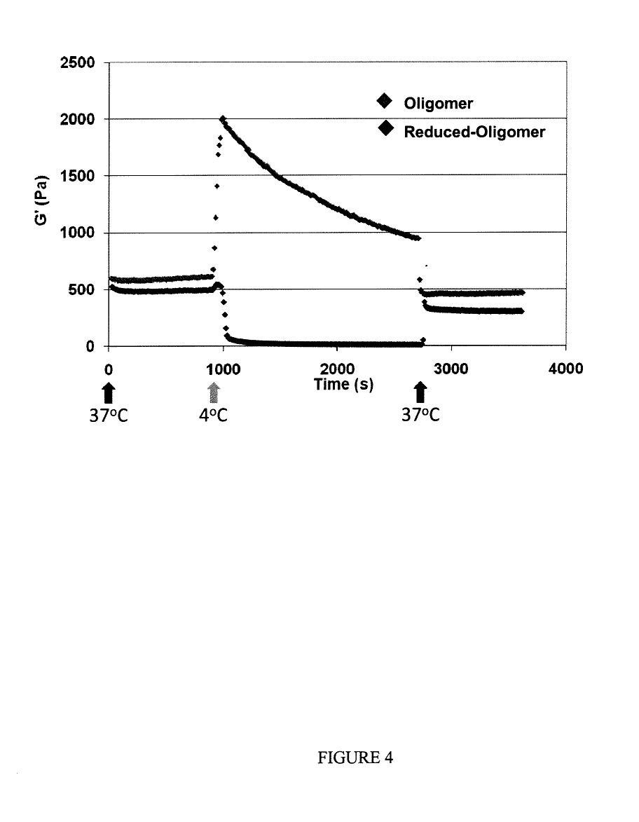

To evaluate time-dependent changes in shear storage modulus (G′) for oligomer and reduced-oligomer collagens in response to temperature modulation between 37° C. and 4° C., neutralized collagen solutions (3 mg/ml) were polymerized at 37° C. (t=0 minutes) on the platform of a TA AR2000 rheometer. G′ increased as the collagens transitioned from a solution to fibril-based matrix. Upon cooling to 4° C. (t=15 minutes), reduced-oligomer experienced matrix-to-solution transition as indicated by the rapid decrease in G′ to near zero values (bottom line in FIG. 4). In contrast, the oligomer showed an increase in G′ indicating that the matrix was becoming stiffer with cooling (top line in FIG. 4). Upon rewarming to 37° C. (t=45 minutes), reduced-oligomer transitioned back to matrix form (temperature=37° C.). In contrast, the oligomer showed a decrease in G′ (temperature=37° C.) upon transition from 4° C. to 37° C. Time-dependent changes in shear storage modulus (G′) for oligomer and reduced-oligomer collagens in response to temperature modulation between 37° C. and 4° C. are shown in FIG. 4.

### Example 17

### Monomer and Reduced-Monomer Collagen Changes in Shear Storage Modulus

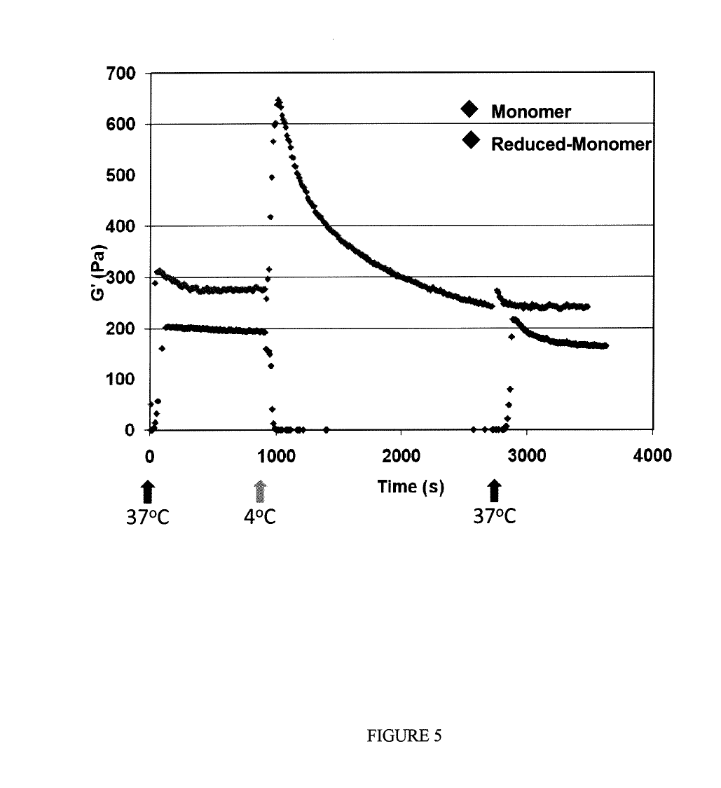

To evaluate time-dependent changes in shear storage modulus (G′) for monomer and reduced-monomer collagens in response to temperature modulation between 37° C. and 4° C., neutralized collagen solutions (3 mg/ml) were polymerized at 37° C. (t=0 minutes) on the platform of a TA AR2000 rheometer. G′ increased as the collagens transitioned from a solution to fibril-based matrix. Upon cooling to 4° C. (t=15 minutes), reduced-monomer experienced matrix-to-solution transition as indicated by the rapid decrease in G′ to near zero values. In contrast, the monomer showed an increase in G′ indicating that the matrix was showing a transient increase in stiffness with cooling. Upon rewarming of samples to 37° C. (t=45 minutes), reduced-monomer transitioned back to matrix form (temperature=37° C.). In contrast, the monomer showed a decrease in G′ (temperature=37° C.) upon transition from 4° C. to 37° C. The phase transition times for monomer and reduced-monomer collagens was slightly greater than those observed for their oligomer counterparts. FIG. 5 shows time-dependent changes in shear storage modulus (G′) for monomer and reduced-monomer collagens in response to temperature modulation between 37° C. and 4° C.

### Example 18

### Atelo-Collagen Changes in Shear Storage Modulus

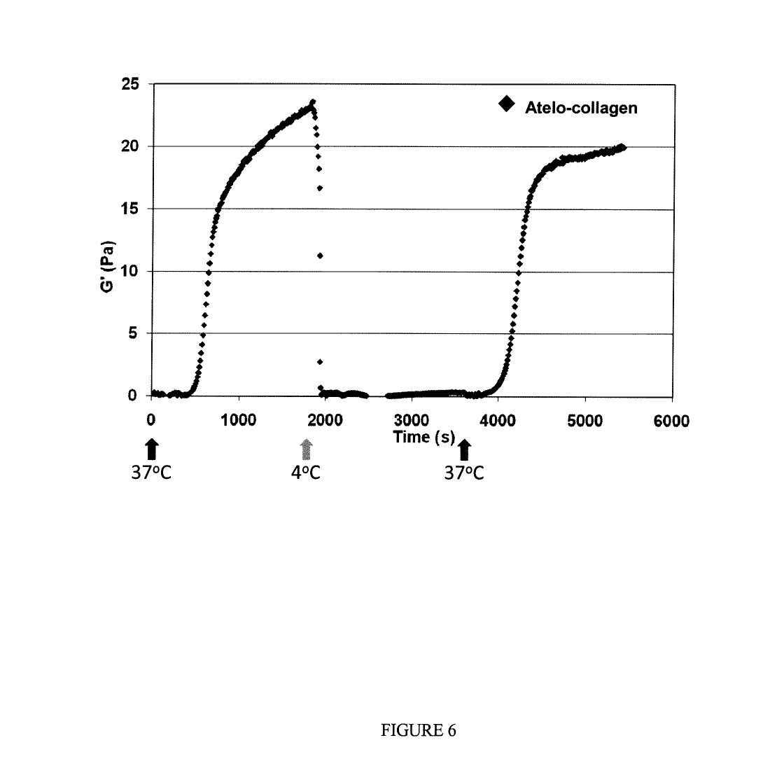

To evaluate time-dependent changes in shear storage modulus (G′) for atelo-collagen in response to temperature modulation between 37° C. and 4° C., atelo-collagen (3 mg/ml) was polymerized at 37° C. (t=0 minutes) on the platform of a TA AR2000 rheometer. G′ increased as atelo-collagen transitioned from a solution to fibril matrix. Upon cooling to 4° C. (t=30 minutes), atelo-collagen experienced matrix-to-solution transition marked by a decrease in G′. Upon rewarming of the sample to 37° C. (t=60 minutes), atelo-collagen transitioned back to matrix form (temperature=37° C.). The phase transition time for atelo-collagen was greater than those observed for reduced forms of oligomer and monomer collagens. Time-dependent changes in shear storage modulus (G′) for atelo-collagen in response to temperature modulation between 37° C. and 4° C. are shown in FIG. 6.

### Example 19

### Oligomer and Reduced-Oligomer Collagen Changes in Absorbance

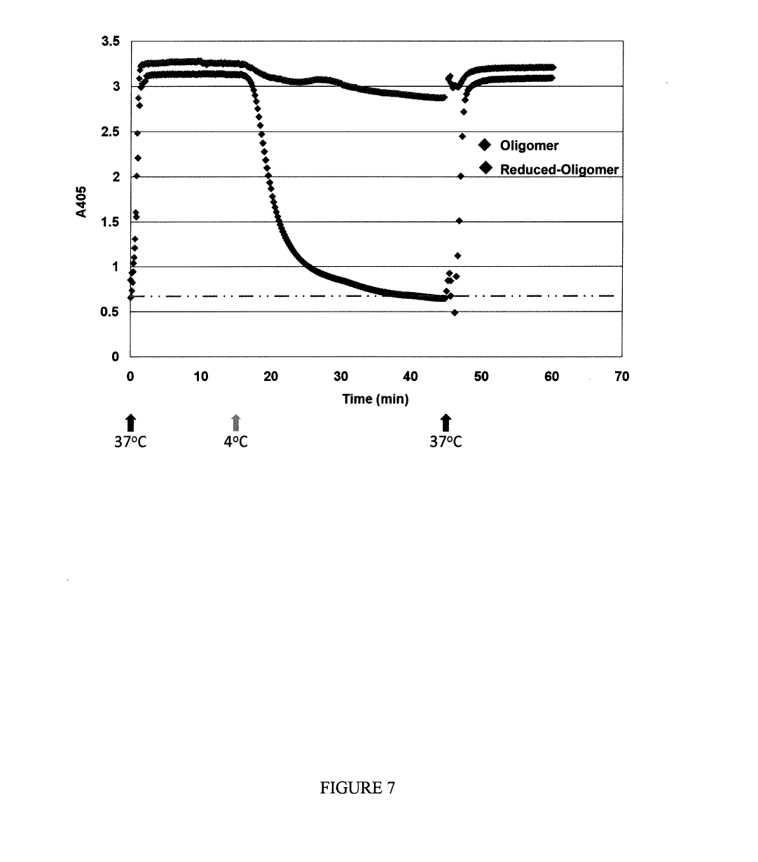

To evaluate time-dependent changes in absorbance at 405 nm (A405) for oligomer and reduced-oligomer collagens in response to temperature modulation between 37° C. and 4° C., neutralized collagen solutions (3 mg/ml) were polymerized at 37° C. (t=0 minutes) as indicated by the increase in turbidity. Upon cooling to 4° C. (t=15 minutes) the reduced-oligomer experienced matrix-to-solution transition as indicated by the rapid decrease in A405 to t=0 baseline values. Rewarming of the reduced-oligomer to 37° C. (t=45 minutes) induced a solution-to-matrix transition (temperature=37° C.). In contrast, the oligomer showed little to no change in A405 throughout the cooling and rewarming cycles. Time-dependent changes absorbance at 405 nm (A405) for oligomer and reduced-oligomer collagens in response to temperature modulation between 37° C. and 4° C. are shown in FIG. 7.

### Example 20

### Monomer and Reduced-Monomer Collagen Changes in Absorbance

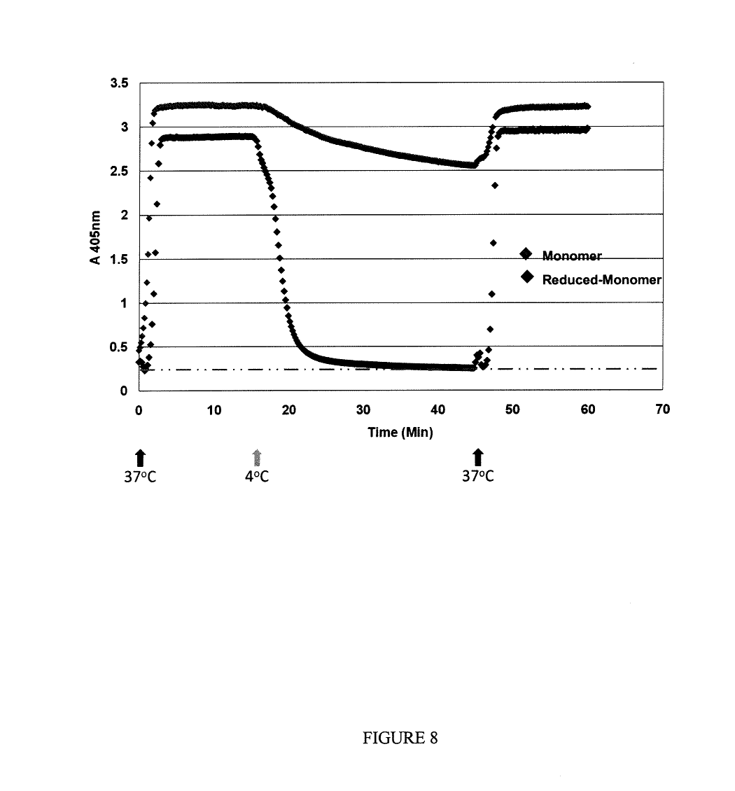

To evaluate time-dependent changes in absorbance at 405 nm (A405) for monomer and reduced-monomer collagens in response to temperature modulation between 37° C. and 4° C., neutralized collagen solutions (3 mg/ml) were polymerized at 37° C. (t=0 minutes) as indicated by the increase in turbidity. Upon cooling to 4° C. (t=15 minutes) the reduced-monomer experienced matrix-to-solution transition as indicated by the rapid decrease in A405 to t=0 baseline values. Rewarming of reduced-monomer to 37° C. (t=45 minutes) induced a solution-to-matrix transition (temperature=37° C.). In contrast, the monomer showed only a gradual and slight decrease in A405 in response to the cooling and rewarming cycles. Reduced-monomer showed a slightly greater phase transition time compared to reduced-oligomer. Time-dependent changes absorbance at 405 nm (A405) for monomer and reduced-monomer collagens in response to temperature modulation between 37° C. and 4° C. are shown in FIG. 8.

### Example 21

### Atelo-Collagen Changes in Absorbance

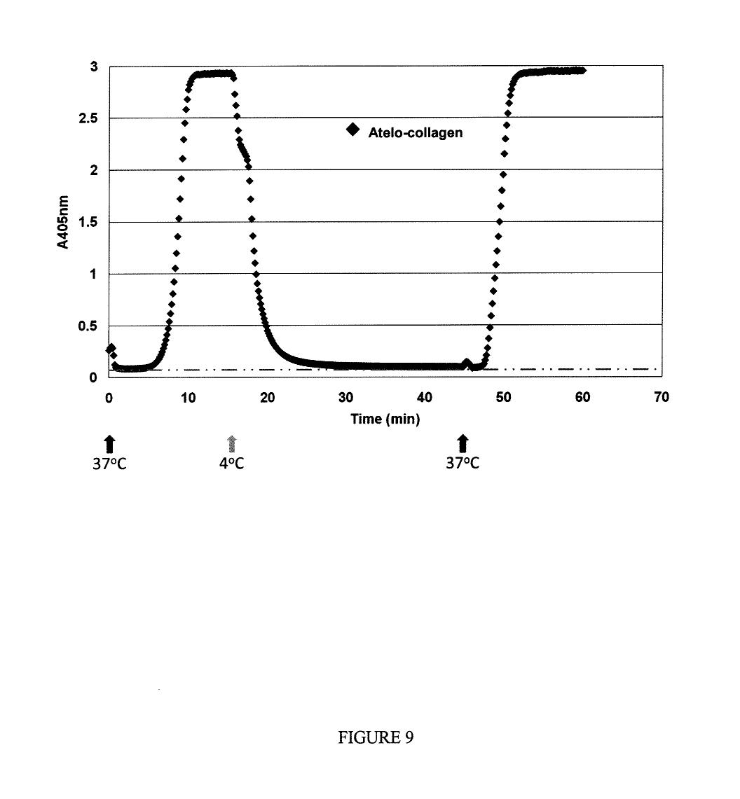

To evaluate time-dependent changes in absorbance at 405 nm (A405) for atelo-collagen in response to temperature modulation between 37° C. and 4° C., neutralized atelo-collagen (3 mg/ml) was polymerized at 37° C. (t=0 minutes) as indicated by the increase in turbidity. Upon cooling to 4° C. (t=15 minutes), atelo-collagen experienced matrix-to-solution transition as indicated by the rapid decrease in A405 to t=0 baseline values (dashed line). Rewarming atelo-collagen to 37° C. (t=45 minutes) induced a solution-to-matrix transition (temperature=37° C.). The phase transition time for atelo-collagen was greater than those observed for reduced forms of oligomer and monomer collagens. Time-dependent changes absorbance at 405 nm (A405) for atelo-collagen in response to temperature modulation between 37° C. and 4° C. are shown in FIG. 9.

### Example 22

### Polymerization Kinetic Parameters of Various Collagen Formulations

Table 1 shows polymerization kinetic parameters for the various collagen formulations. Each collagen was neutralized under the same reaction conditions and collagen concentration (3 mg/ml). Polymerization kinetic parameters were determined using a well-established turbidity assay.

TABLE 1
Polymerization Kinetic Parameters of Various Collagen Formulations

| | | Lag Phase | Half- |
|---|---|---|---|
| Collagen | Growth Phase Rate | Duration | Polymerization |
| Formulation | (ΔA405/sec) | (sec) | Time (sec) |
| Oligomer | 0.032 | 0 | 43.10 |
| Reduced-oligomer | 0.017 | 0 | 47.77 |
| Monomer | 0.027 | 13.44 | 72.68 |
| Reduced-monomer | 0.035 | 83.15 | 125.93 |
| Atelo-collagen | 0.016 | 426.54 | 520.79 |

### Example 23

### LSK Proliferation within Various Collagen Culture Parameters

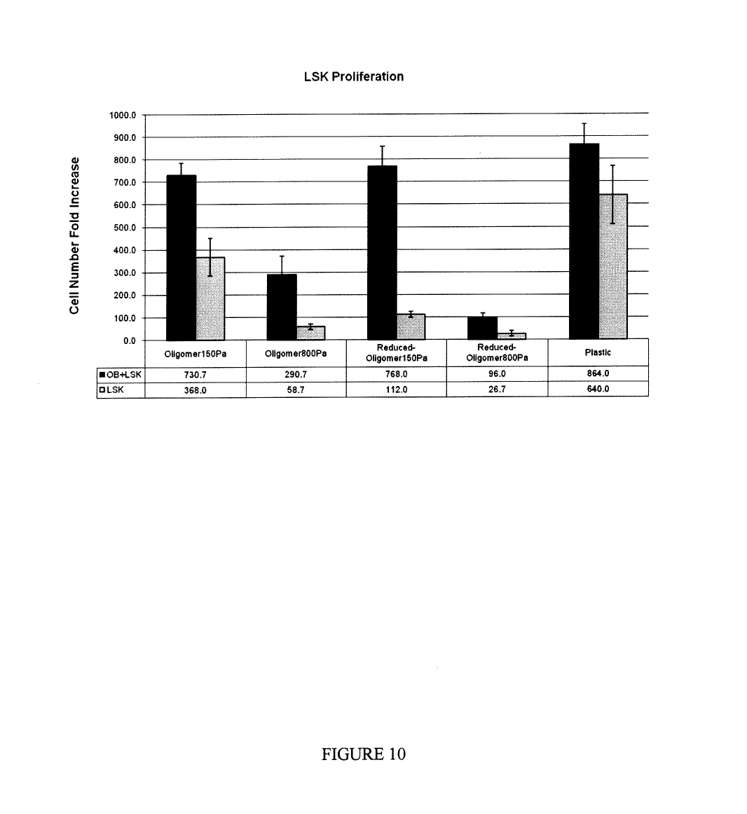

FIG. 10 shows a comparison of LSK proliferation within various collagen culture parameters. Statistical comparison between groups used a two-tail t-test assuming unequal variances (a p-value less than 0.05 was considered statistically significant).

LSK cultured alone within 3D collagen matrices showed decreased proliferation compared to those on plastic (p<0.05 for all groups except Oligomer150 Pa, where p=0.054). Co-culture of LSK in the presence of OB significantly enhanced (p<0.05) LSK proliferation within all 3D matrix formulations. While an increase in LSK proliferation was noted for LSK+OB cultures on plastic, it was not statistically significant (p=0.069).

LSK proliferation was statistically similar for LSK+OB cultures on plastic and both 3D matrix formulations at 150 Pa. In contrast, LSK proliferation within high stiffness Oligomer 800 Pa and Reduced-Oligomer 800 Pa matrices decreased by 3- and 9-fold, respectively, compared to that on plastic.

LSK proliferation was statistically similar (p>0.05) for OB+LSK co-cultures within oligomer and reduced-oligomer formulations at each of the stiffness values tested. At a given matrix stiffness, LSK alone cultures proliferated significantly less (p<0.05) within reduced-oligomer compared to oligomer.

### Example 24

### LSK Viability within Various Collagen Culture Parameters

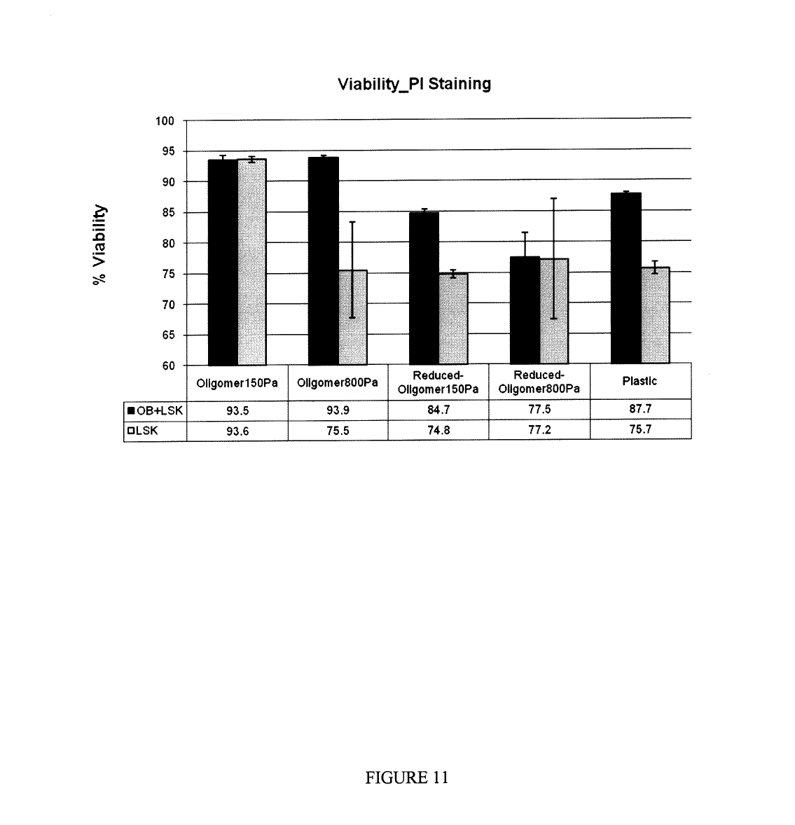

FIG. 11 shows a comparison of LSK viability within various collagen culture parameters. Viability of LSK cultured alone was greatest for oligomer150 Pa at 93.6±0.8%. LSK viability when cultured alone on all other 3D matrices was roughly 75% and statistically similar (p>0.05) to that observed on plastic. Viability stayed the same or was enhanced when LSK were cultured with OB in any format tested. Viability of OB+LSK cultures was greatest for oligomer150 Pa and oligomer800 Pa matrices.

### Example 25

### Percentage of LSK Lin−Sca1+ Cells within Various Collagen Culture Parameters

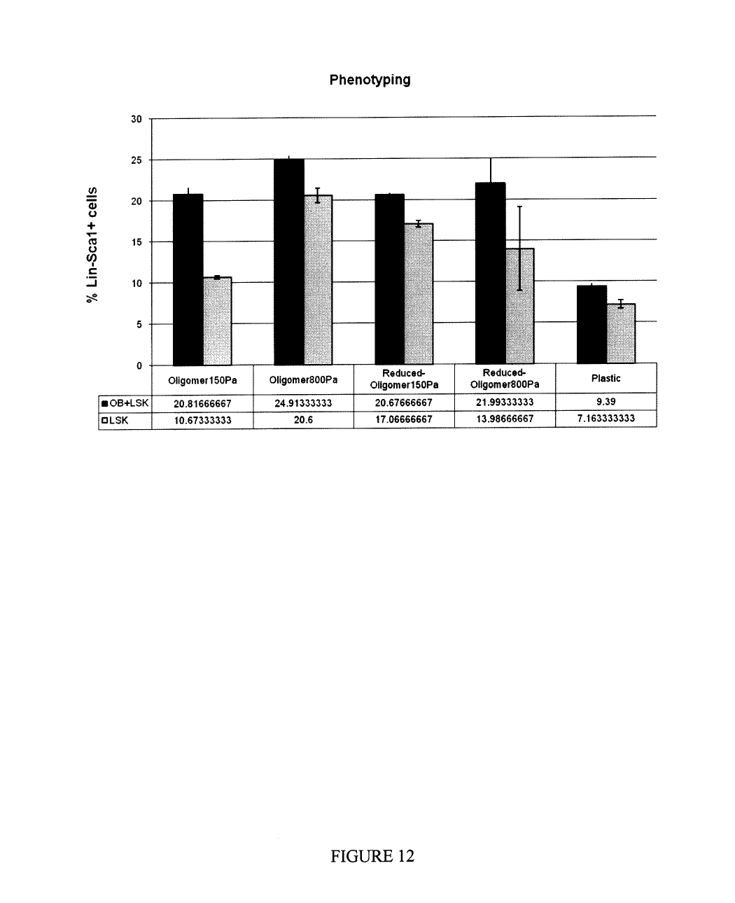

FIG. 12 shows a comparison of the percentage of LSK Lin−Sca1+ cells within various collagen culture parameters. LSK cultured alone or with OB within 3D collagen matrices showed an increased percentage of Lin−Sca1+ cells compared to those on plastic. In all cases the increase relative to plastic was statistically significant (p<0.05) with the exception of reduced-oligomer800 Pa, which showed a high degree of variability and p=0.1064. In general, culturing LSK in the presence of OB enhanced the percentage of Lin−Sca1+ (p<0.05 except reduced-oligomer800 Pa, where p=0.064).

For OB+LSK cultures there was no different in the percentage of Lin−Sca1+ between matrix formulations of the same stiffness. The percentage of Lin−Sca1+ cells was statistically higher for LSK alone and OB+LSK when cultured in oligomer800 Pa compared to oligomer150 Pa. In contrast, stiffness had no effect on percentage of Lin−Sca1+ for cells cultured reduced-oligomer.

### Example 26

### Confocal Reflection Images of Oligomer and Reduced-Oligomer Matrices

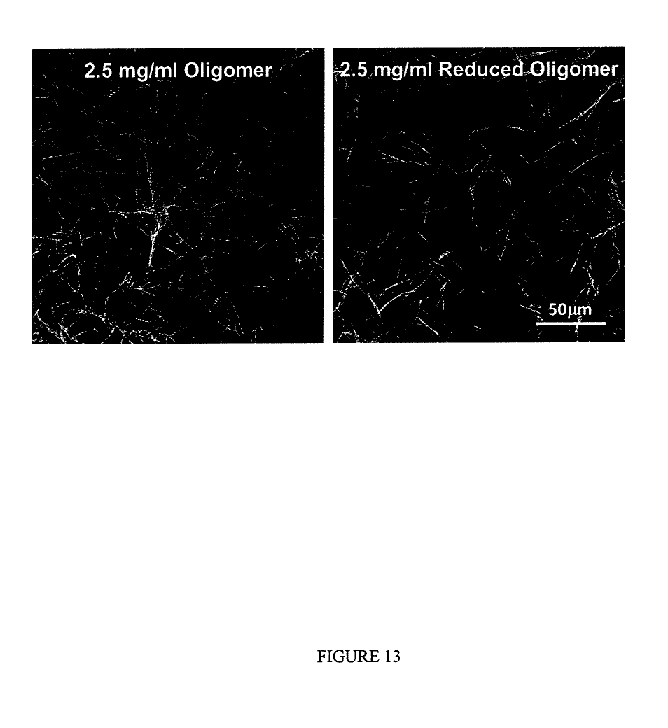

Confocal reflection images showing single slice (1 um) view of collagen-fibril microstructure of oligomer and reduced-oligomer matrices are shown in FIG. 13. Matrices were polymerized under the same conditions at 2.5 mg/ml collagen concentrations. The observed differences in fibril microstructure, stiffness (G′), and thermal properties suggest that the intermolecular cross-links formed by reactive aldehydes within oligomer formulations contribute to increased matrix mechanical integrity as well as increased thermal stability.

### Example 27

### Thermo-Responsive Behavior of Thermoreversible and Unmodified Collagen Formulations

The temperature-induced change in collagen physico-chemical properties can be readily demonstrated using traditional rheometric or spectrophotometric approaches. Neutralized collagen solutions (same collagen concentration) were polymerized at 37° C. on the platform of a TA AR2000 rheometer. Samples were tested in oscillatory shear and the temperature was adjusted to 4° C. and then back to 37° C. The modified type I collagen reversibly transitions from a fibril-based matrix to a solution form upon cooling to 4° C. In contrast, the behavior of the control collagen formulation is dominated by the insoluble fibril component despite the fluctuation in temperature.

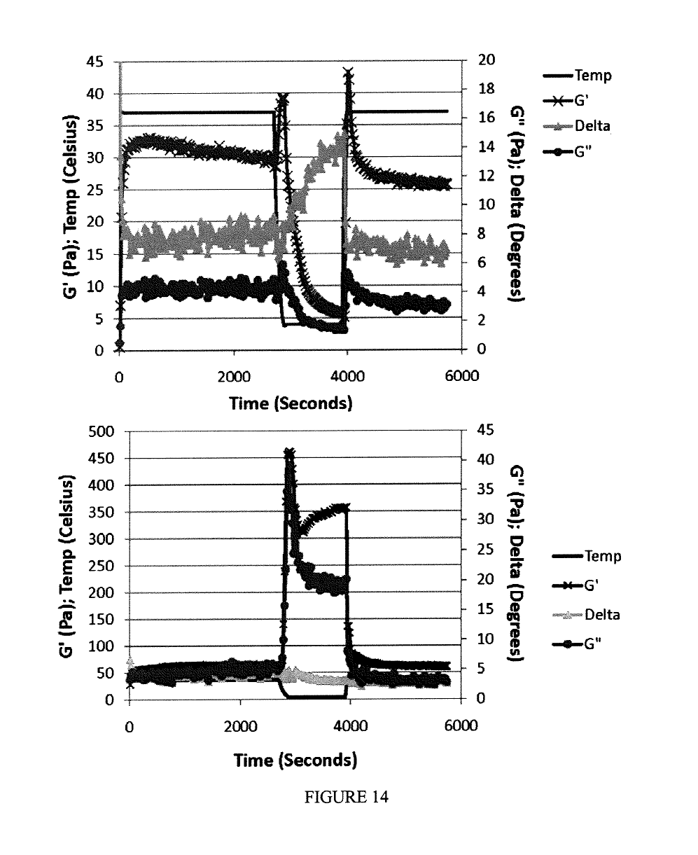

FIG. 14 compares the thermo-responsive behavior of both thermoreversible and unmodified control collagen formulations. In both cases, the collagen formulations were allowed to polymerize on a temperature-controlled platen of a rheometer and tested in oscillatory shear as temperature was transitioned between 37° C. and 4° C. When polymerized at similar concentrations and conditions, the thermoreversible collagen has moderately less mechanical integrity (G′=33 Pa; Delta=8o) compared to that of control collagen (G′=64 Pa; Delta=4o). However, in both cases, the viscoelastic properties of the polymerized matrices were obviously dominated by the insoluble fibril component. When temperature is decreased from 37° C. to 4° C., thermoreversible collagen showed a rapid decline in G′ and a concomitant increase in delta indicative of more viscous, fluid-like behavior. Reheating the sample to 37° C. showed its thermal memory as G′ and delta returned to their original values. In contrast, the unmodified control collagen showed no such matrix to solution phase transition upon cooling. In this case, there was an observed increase in G′ and a moderate decrease in delta, indicating that the matrix showed enhanced elastic solid-like behavior and decreased fluidity upon cooling to 4° C.

### Example 28

### Physical Properties of Thermoreversible and Unmodified Collagen Formulations

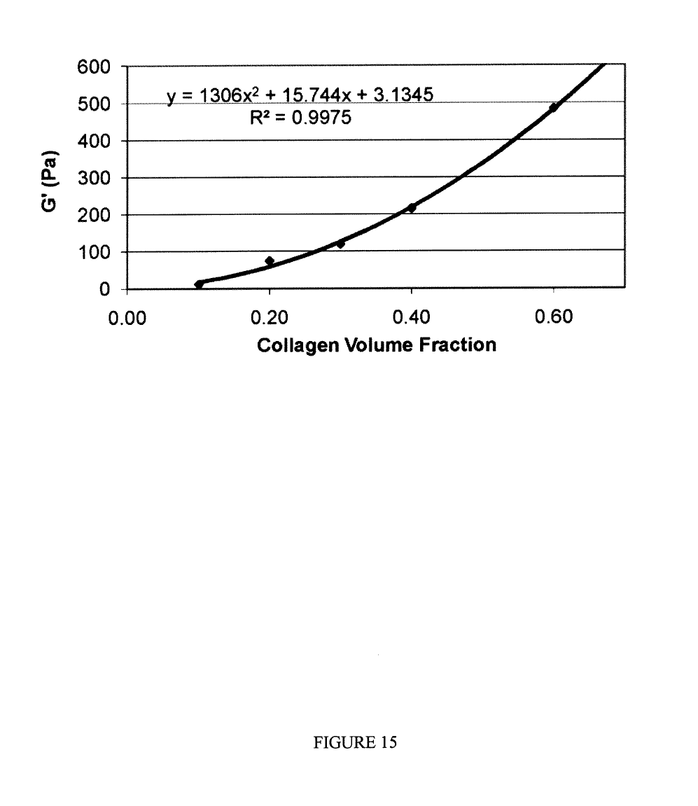

The physical properties (e.g., matrix stiffness, degradation) of the resultant thermoreversible matrix can be controlled by varying relevant polymerization parameters (e.g., collagen concentration, oligomer/monomer content, oligomer type) as previously described for standard purified collagen formulations. FIG. 15 shows the modulation of G′ for a thermoreversible collagen matrix as collagen concentration (collagen volume fraction) is varied. In addition, the biochemical composition of the matrix can be readily modified by adding soluble factors, drugs, and other molecules prior to polymerization.

### Example 29

### Application of Thermoreversible Collagen for Continuous 3D Culture or Cryopreservation of Cells

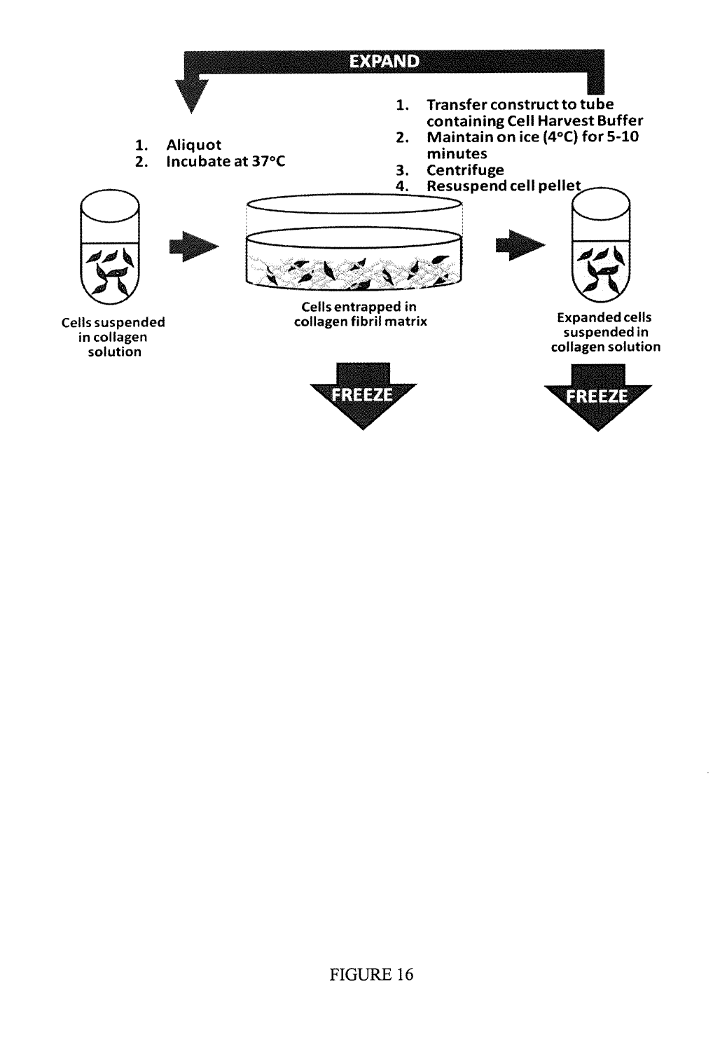

The application of thermoreversible collagen for continuous 3D culture or cryopreservation of cells was evaluated. Three different cell types, including a lymphoblast cell line (VM-2, ATTC), neonatal human dermal fibroblasts (NHDF, Lonza), and human endothelial progenitor cells derived from umbilical artery (EPC-Artery, Merv Yoder) were seeded at 5×10⁵ cells/ml within the thermoreversible collagen. These three cell types were chosen based upon their presumed differences in cell-matrix adhesion, collagen production, and matrix remodeling potential (VM-2<NHDF<EPC). As shown in FIG. 16, cells were harvested from the matrices at 2, 24, and 48 hours. Cells are suspended in a neutral collagen solution. Polymerization of the collagen with cell entrapment is induced upon warming to 37° C. Cells can be readily removed by cooling the construct to 4° C. to induce matrix to solution transition. Recovered cells may be analyzed or resuspended in thermoreversible collagen for continued propagation. Cells may be cryopreserved when entrapped as part of an intact 3D tissue construct or when suspended in a collagen solution.

### Example 30

### Recovery Efficiency and Viability of Cells

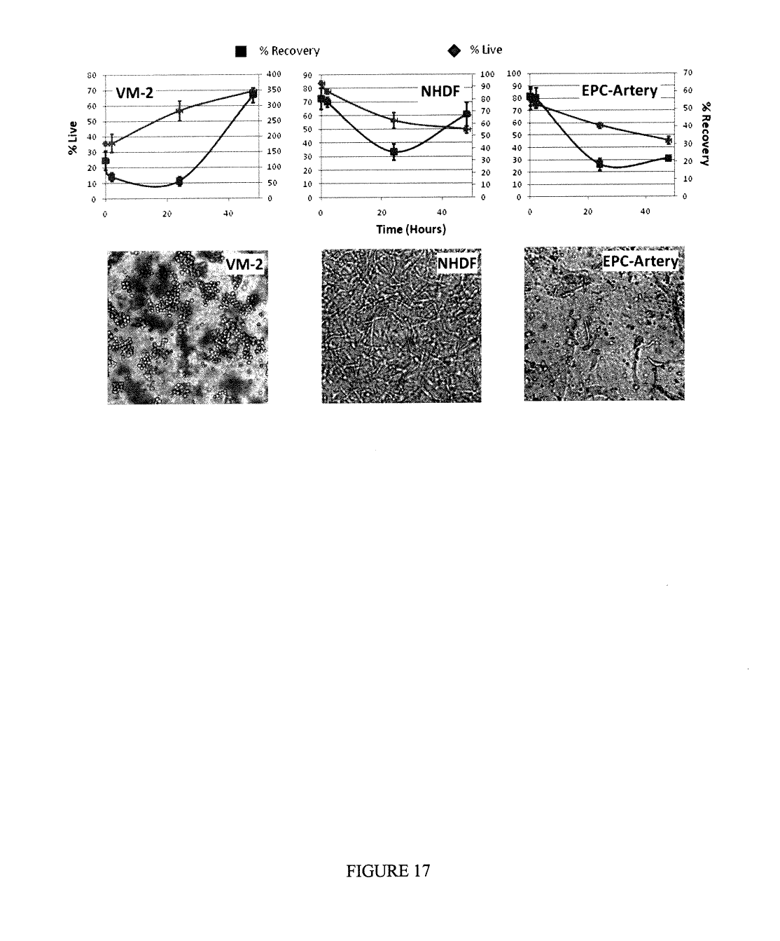

As shown in the upper panel of FIG. 17, the recovery efficiency and viability of cells were determined. Cells were seeded within the matrix (5×10⁵ cells/ml) and maintained within cell-specific complete medium. At 2, 24, and 48 hours, constructs were cooled to induce matrix to solution transition and cells recovered in Cell Harvest Buffer. Timepoint 0 represents the original cell population subjected to the cell harvesting protocol. Live/dead counts were performed using a hemocytometer. Light micrographs of 3D cell cultures were obtained after 48 hours and are shown in the bottom panel of FIG. 17.

The three cell types as described in Example 29 showed different responses (e.g., morphology, proliferation) and harvest efficiencies. Surprisingly, VM-2, which are routinely propagated in suspension, showed a dramatic increase in cell number as well as an improvement in viability over the 48-hour time period. After 48 hours, cell recovery was 3.5 times higher than the number originally seeded within the matrix. Furthermore, during this time cell viability increased from 35.7% at the time of seeding to 69.1% at 48 hours. NHDF showed a significant decline in viability between 2 and 24 hours. However, no significant difference in viability was observed between 24 and 48 hours. These cells took on a spindle shape and increased in number over time as indicated by the increase in recovery percentage between 24 and 48 hours. EPC, which are known to undergo vacuolization and vessel formation within collagen matrices, showed little to no vessel formation and limited vacuolization. EPC viability showed a steady decline over the time period study, while recovery decreased significantly between 2 and 24 hours and then appeared to stabilize at around 20%. A significant number of multi-cellular EPC structures were observed adhered to the bottom of the culture well-plate.

What is claimed is:

1. A composition comprising an engineered collagen matrix wherein the matrix comprises sterilized thermoreversible collagen.

2. The composition of claim 1 further comprising cells.

3. The composition of claim 1 wherein the thermoreversible collagen consists essentially of reduced collagen monomers.

4. The composition of claim 1 wherein the thermoreversible collagen comprises reduced collagen oligomers.

5. The composition of claim 4 wherein the percentage of collagen oligomers based on total isolated collagen in dry weight/ml used to make the matrix is about 10 percent or greater.

6. The composition of claim 2 wherein the cells are hematopoietic stem cells.

7. The composition of claim 1 wherein the composition is in a sterile package.

8. The composition of claim 7 wherein the composition is a medical graft.

9. The composition of claim 4 wherein the composition is in a sterile package and wherein the composition is a medical graft.

10. A method for isolating cells for implantation into a patient, the method comprising the steps of,
polymerizing thermoreversible collagen to form an engineered collagen matrix;
contacting the thermoreversible collagen with the cells before or after polymerizing the thermoreversible collagen to form the matrix;
proliferating the cells or maintaining the viability of the cells;
depolymerizing the matrix comprising thermoreversible collagen;
separating the cells from the depolymerized thermoreversible collagen; and
implanting or injecting the cells into the patient.

11. The method of claim 10 wherein the thermoreversible collagen consists essentially of reduced collagen monomers.

12. The method of claim 10 wherein the thermoreversible collagen comprises reduced collagen oligomers.

13. The method of claim 12 wherein the percentage of collagen oligomers based on total isolated collagen in dry weight/ml used to make the matrix is about 10 percent or greater.

14. The method of claim 10 wherein the cells are hematopoietic stem cells.

15. The method of claim 10 wherein the cells are used as an injectable or implantable composition for wound healing, a bone marrow transplant, or for cosmetic surgery.

16. The method of claim 10 wherein the polymerization occurs in response to heating to a temperature that causes a collagen solution to matrix transition and wherein the depolymerization occurs in response to cooling to a temperature that causes a collagen matrix to solution transition.

17. A method for treating a patient with diseased or damaged tissues, the method comprising the step of implanting or injecting thermoreversible collagen into the patient.

18. The method of claim 17 wherein the thermoreversible collagen consists essentially of reduced collagen monomers.

19. The method of claim 17 wherein the thermoreversible collagen comprises reduced collagen oligomers.

20. The method of claim 17 wherein the thermoreversible collagen is in the form of an injectable or an implantable medical graft for wound healing or for cosmetic surgery.
